(19) United States
(12) Patent Application Publication
(10) Pub. No.: US 2025/0099341 A1
(43) Pub. Date: Mar. 27, 2025

(54) SYSTEMS, APPARATUS, AND METHODS FOR PLACING AND/OR CONFIRMING A LOCATION OF AN END OF A NASOGASTRIC OR AN OROGASTRIC TUBE

(71) Applicant: CoapTech, Inc., Baltimore, MD (US)

(72) Inventors: Steven P. TROPELLO, Baltimore, MD (US); William Alexander LeMay PATTERSON, Dundalk, MD (US); Michael Stuart ZIMMERMAN, JR., Cockeysville, MD (US); Noah BARASCH, Brooklyn, NY (US); Carly Marie CLAYTON, Mount Airy, MD (US); Sean Michael CZAJA, Sacramento, CA (US)

(21) Appl. No.: 18/761,866

(22) Filed: Jul. 2, 2024

Related U.S. Application Data
(63) Continuation-in-part of application No. PCT/US2023/060313, filed on Jan. 9, 2023.
(60) Provisional application No. 63/297,457, filed on Jan. 7, 2022.
(60) Provisional application No. 63/562,474, filed on Mar. 7, 2024.

(51) Int. Cl. A61J 15/00; A61M 25/01; A61M 25/10

(52) U.S. Cl. CPC A61J 15/0084; A61J 15/0003; A61J 15/0049; A61M 25/0127; A61M 2025/0166; A61M 25/1018; A61M 2205/581

(57) ABSTRACT

Systems, apparatus, and methods for placing and/or confirming a location of an end of a nasogastric or an orogastric tube within a body cavity are described herein. In some embodiments, an apparatus includes an elongated tube and a position indicator. The elongated tube has a first end, a second end, and defines a lumen. The position indicator is coupled to the elongated tube and disposed sufficiently close to the first end such that, when the first end is properly disposed in the cavity of the patient, the position indicator is disposed in the cavity of the patient. The position indicator is configured to produce an indication perceivable outside of the patient that the position indicator is disposed in the cavity of the patient. The indication is an indication differentiable from a sound naturally generated by the cavity of the patient.

### CROSS-REFERENCE TO RELATED APPLICATIONS

This application is a continuation-in-part of International Patent Application No. PCT/US2023/060313, filed Jan. 9, 2023, entitled “Systems, Apparatus, and Methods for Placing and/or Confirming a Location of an End of a Nasogastric or an Orogastric Tube,” which claims priority to and the benefit of U.S. Provisional Application No. 63/297,457, filed Jan. 7, 2022, entitled “Systems, Apparatus, and Methods for Placing and/or Confirming a Location of an End of a Nasogastric or an Orogastric Tube,” and this application claims priority to and the benefit of U.S. Provisional Application No. 63/562,474, filed Mar. 7, 2024, entitled “Systems, Apparatus, and Methods for Placing and/or Confirming a Location of an End of a Nasogastric or an Orogastric Tube,” the entire contents of each of which are incorporated by reference herein for all purposes.

### BACKGROUND

Embodiments described herein relate to systems, apparatus, and methods for placing and/or confirming a location of an end of a nasogastric or an orogastric tube within a body cavity.

Delivering a medical device, such as an end of a nasogastric or an orogastric tube, to a location within a patient (e.g., a stomach) may include guiding the medical device through a bifurcated body lumen. Such procedures are sometimes performed blindly, risking misplacement of the medical device within the patient's body and/or injury to the patient's body. Furthermore, delivery of a medical device through a lumen of a patient can be even more difficult in high risk patients (e.g., sedated patients, endotracheally-intubated patients, and/or agitated patients). Additionally, after proper initial placement of an end of a nasogastric or an orogastric tube in a stomach of a patient, the nasogastric or orogastric tube may migrate relative to the patient's stomach (e.g., into the patient's esophagus or jejunum). Thus, caregivers, such as nurses, need to check the nasogastric or orogastric tube periodically (e.g., every four hours) to confirm that the end of the nasogastric or orogastric tube has not migrated unsafely.

One typical method of monitoring the location of the end of the nasogastric or orogastric tube includes marking and tracking a location of a portion of the tube external to the patient relative to a nasal or oral orifice through which the tube passes, but such a method may not indicate that the end has migrated into the esophagus (e.g., via being refluxed up) or traveled into the trachea, which could be catastrophic. Another method of monitoring the location of the end of the nasogastric or orogastric tube includes delivering a volume of air through the tube and into the stomach and listening for a gurgle sound (sometimes referred to as a “whoosh” or “blow” test), but such a gurgle sound is easily confused with other stomach noises. Another approach is to perform a pH test on aspirate fluid drawn through the tube using pH indicator paper to confirm that the aspirate fluid is stomach acid, but such an approach has been shown to be unreliable due to other substances in contact with the tube prior to and/or during insertion of the end of the tube into the stomach and/or user error. Another known approach includes using an enteral access system including electromagnetic sensors in a stylet for localization of a tip of a tube during tube insertion, but such an approach has been associated with pneumothorax events requiring urgent intervention (e.g., needle decompression or chest tube placement), and has sometimes been associated with cardiopulmonary arrest and patient death. While the location of the tube within the patient can be confirmed via X-ray imaging, X-ray imaging requires the use of additional equipment and the involvement of specialized clinicians to read the imaging and carries the risk of radiation-induced injuries.

Thus, there is a need for systems, apparatus, and methods for confirming delivery of an end of a nasogastric or orogastric tube beyond a bifurcation in a body lumen and confirming location of the end of the tube at periodic intervals, which reduce risks to the patient and allow for the medical device to be quickly and easily placed in its intended location.

### SUMMARY

Systems, apparatus, and methods for placing and/or confirming a location of an end of a nasogastric or an orogastric tube within a body cavity are described herein. In some embodiments, an apparatus includes an elongated tube and a position indicator. The elongated tube has a first end, a second end, and defines a lumen extending from the second end to a location at or near the first end of the elongated tube. The elongated tube defines at least one opening in fluid communication with the lumen such that, when the first end of the elongated tube is disposed in a cavity of a patient and the second end of the elongated tube is disposed outside of the patient, fluid can be delivered from the second end of the elongated tube into the cavity via the lumen and the at least one opening. The position indicator is coupled to the elongated tube and disposed sufficiently close to the first end such that, when the first end is properly disposed in the cavity of the patient, the position indicator is disposed in the cavity of the patient. The position indicator is configured to produce an indication perceivable outside of the patient that the position indicator is disposed in the cavity of the patient. The indication is an indication differentiable from a sound naturally generated by the cavity of the patient.

### BRIEF DESCRIPTION OF THE DRAWINGS

FIG. 1 is a schematic illustration of a tube disposed in a patient, according to an embodiment.

FIG. 2 is a schematic illustration of a system, according to an embodiment.

FIGS. 3-11 are various views of a system and portions of the system, according to an embodiment.

FIGS. 12 and 13 are a perspective view and a side view of a system, according to an embodiment.

FIGS. 14-17 are various views of a portion of a system, according to an embodiment.

FIG. 18 is a flow chart illustrating a method of using any of the systems and/or assemblies described herein, according to an embodiment.

FIG. 19 is a flow chart illustrating a method of using any of the systems and/or assemblies described herein, according to an embodiment.

FIG. 20 is a schematic illustration of a system, according to an embodiment.

FIG. 21 is a flow chart illustrating a method of using any of the systems and/or assemblies described herein, according to an embodiment.

FIGS. 22-29 are schematic illustrations of a visual indicator, according to an embodiment.

FIGS. 30-35 are schematic illustrations of various portions of a system, according to an embodiment.

### DETAILED DESCRIPTION

Systems, apparatus, and methods for confirming placement and location of a medical device or a portion (e.g., an end) of a medical device in a location within a patient's body are described herein. The medical device may include, for example, a tube such as a nasogastric tube and/or an orogastric tube. During delivery of the medical device to the location within the patient's body, the medical device may need to navigate or be guided beyond a bifurcation in a body lumen of the patient's body such that the medical device is advanced through an intended passageway and avoids an unintended route. Bifurcated body lumens may be encountered, for example, when a medical device is delivered through an esophagus to a patient's stomach, avoiding the patient's trachea and lungs. Additionally, after the initial placement of the medical device, confirmation of a location of a portion of the medical device (e.g., an end) can be performed at periodic intervals to ensure that the portion of the medical device has not migrated unsafely within the patient's body.

Systems, apparatus, and methods for placing and/or confirming a location of an end of a nasogastric or an orogastric tube within a body cavity are described herein. In some embodiments, an apparatus includes an elongated tube and a position indicator. The elongated tube has a first end, a second end, and defines a lumen extending from the second end to a location at or near the first end of the elongated tube. The elongated tube defines at least one opening in fluid communication with the lumen such that, when the first end of the elongated tube is disposed in a cavity of a patient and the second end of the elongated tube is disposed outside of the patient, fluid can be delivered from the second end of the elongated tube into the cavity via the lumen and the at least one opening. The position indicator is coupled to the elongated tube and disposed sufficiently close to the first end such that, when the first end is properly disposed in the cavity of the patient, the position indicator is disposed in the cavity of the patient. The position indicator is configured to produce an indication perceivable outside of the patient that the position indicator is disposed in the cavity of the patient. The indication is an indication differentiable from a sound naturally generated by the cavity of the patient.

In some embodiments, a method includes producing a fluid flow through a lumen of an elongated tube such that the fluid flow interacts with a position indicator coupled to the elongated tube and disposed within a patient. The method further includes determining whether the position indicator generated an indication that the position indicator is disposed within a target cavity of the patient in response to the fluid flow, the indication being an indication differentiable from a sound naturally generated by the cavity of the patient.

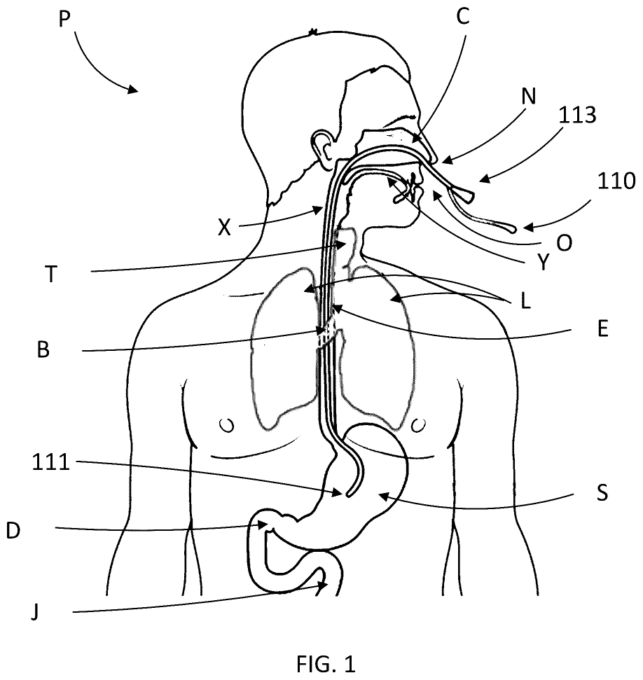

FIG. 1 is a schematic illustration of an elongated tube 110 partially disposed in a patient P. An end of the elongated tube 110 may be delivered to a patient's stomach such that fluids can be delivered to a stomach S of the patient P through the elongated tube 110. As shown in FIG. 1, the patient P has an oral cavity Y, a nasal cavity C, a pharynx X, an esophagus E, the stomach S, and a small bowel including a duodenum D and a jejunum J. The patient P includes a nasal orifice N that provides access to the nasal cavity C and an oral orifice O that provides access to the oral cavity Y. Thus, a pathway to the stomach S can include the nasal orifice N and nasal cavity C (or the oral orifice O and oral cavity Y), the pharynx X, and the esophagus E.

When a patient P has difficulty swallowing food and/or liquid, the elongated tube 110 (e.g., a nasogastric or an orogastric tube) can be placed via the nasal orifice N or the oral orifice O and the esophagus E of the patient P such that nutrition and/or medication can be provided directly through the tube to the stomach S. For example, as shown in FIG. 1, the elongated tube 110 (e.g., a nasogastric tube) can be inserted through the nasal orifice N, through the nasal cavity C, through the pharynx X, through the esophagus E and into the stomach S. Thus, the elongated tube 110 can have the first end 111 disposed within the stomach S and a second end 113 disposed outside of the patient P, with the elongated tube 110 passing through the patient's nasal orifice N. While the elongated tube 110 is shown as being disposed within the patient's nasal orifice N and nasal cavity C, in some embodiments the elongated tube 110 can be inserted via an oral orifice O of the patient, into the patient's oral cavity Y, into the pharynx X, through the esophagus E, and into the stomach S. Thus, the stomach S can be accessible from the nasal orifice N or the oral orifice O via the pharynx X and esophagus E.

Navigation of the first end 111 of the elongated tube 110 through the patient P, however, can be challenging and pose risks to the patient P. If the first end 111 of the elongated tube 110 is misplaced within the patient's body, such as within a patient's larynx, trachea T, bronchi B (e.g., a patient's bilateral primary bronchi), or lung L, the elongated tube 110 can injure the patient and/or fluid provided through the elongated tube 110 can cause injury to the patient. For example, the elongated tube 110 can cause pulmonary damage, pneumothorax, hemodynamic collapse, and/or pneumonia. Even worse events can occur if the elongated tube 110 is used to introduce medicine or food into the patient's airway. Furthermore, after initial placement of the first end 111 of the elongated tube 110 within the stomach S, the elongated tube 110 may migrate such that the first end 111 is disposed within the duodenum D, the jejunum J, the esophagus E, the larynx, the trachea, or the lung of the patient P.

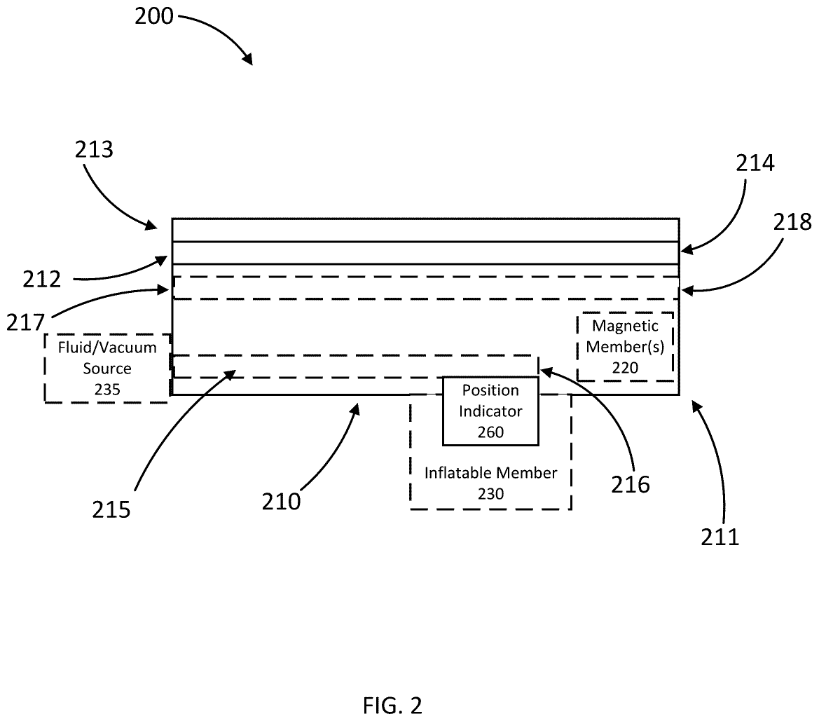

In some embodiments, an elongated tube can include or be coupled to features that aid in navigating a first end of the elongated tube to a stomach of a patient and/or that aid in confirming the location of the first end of the elongated tube within the patient. For example, FIG. 2 is a schematic representation of a system 200. The system 200 includes an elongated tube 210 having a first end 211 and a second end 213 and a position indicator 260 associated with the first end 211 of the elongated tube 210. The elongated tube 210 defines a lumen 212, which can be a feeding lumen. The lumen 212 can extend from the second end 213 to a location at or near the first end 211 of the elongated tube 210. The elongated tube 210 can define at least one opening 214 in fluid communication with the lumen 212 such that, when the first end 211 of the elongated tube 210 is disposed in a cavity of a patient (e.g., a stomach) and the second end 213 of the elongated tube 210 is disposed outside of the patient, fluid can be delivered from the second end 213 of the elongated tube 210 into the cavity via the lumen 212 and the at least one opening 214. In some embodiments, the opening 214 can be defined in the first end 211 of the elongated tube 210 such that fluid can flow through the second end 213, through the lumen 212, and out the opening 214 in the first end 211. In some embodiments, the elongated tube 210 can have one or more openings 214 (also referred to as fenestrations) that can be defined in a sidewall of the elongated tube 210 near the first end 211 such that fluid can flow through the second end 213, through the lumen 212, and out of the one or more fenestrations. The fenestrations can be any suitable size or shape (e.g., rounded, rectangular). In some embodiments, the first end 211 can be closed (e.g., a cap or cover can be coupled to the first end 211) such that fluid communication between the lumen 212 and a body cavity (e.g., the stomach) within which the first end 211 is disposed is only via the one or more fenestrations in the sidewall.

The position indicator 260 can be disposed at or sufficiently close to the first end 211 such that, when the first end 211 of the elongated tube 210 is properly positioned in a stomach of the patient and the second end 213 is disposed outside of the patient (e.g., beyond the patient's nose or mouth), the position indicator 260 is also positioned in the stomach of the patient. The position indicator 260 can be disposed distally to none, one, some, or all of the one or more openings 214 of the elongated tube 210 in communication with the lumen 212. In some embodiments, the position indicator 260 is fluidically isolated from fluid disposed within the lumen 212. In some embodiments, the position indicator 260 is in fluid communication with fluid disposed within the lumen 212. The position indicator 260 is configured to indicate to a user (e.g., a caregiver such as a healthcare practitioner) that the first end 211 of the elongated tube 210 is properly disposed in the stomach of the patient by producing an indication perceivable outside of the patient that the position indicator 260 is disposed in the stomach of the patient. The indication can be an indication differentiable from a sound naturally generated by the cavity of the patient such that the indication is not confused with naturally generated body noises (e.g., stomach or bowel sounds such as gurgling, bubbling, growling, and/or rumbling) and vice versa.

In some embodiments, the indication that can be produced by the position indicator 260 includes an audible indication that the position indicator 260, and thus the first end 211 of the elongated tube 210, is properly disposed in the patient's stomach. The audible indication can be an audible indication that is not a natural sound passively produced by the patient's body (e.g., stomach or bowel sounds such as gurgling, bubbling, growling, and/or rumbling). For example, the audible indication can include a whistle, buzzer, duck call, ring, siren, and/or pop sound. In some embodiments, the position indicator 260 is configured to produce a tactile indication that the position indicator 260, and, thus the first end 211 of the elongated tube 210, is properly and/or improperly disposed in the patient's stomach.

In some embodiments, the position indicator 260 can include a whistle feature. The whistle feature can be in fluid communication with a lumen defined by the elongated tube 210 such that gaseous fluid (e.g., air, nitrogen, carbon dioxide, and/or oxygen) flowing through the lumen can reach the whistle feature and cause the whistle feature to generate a whistle sound sufficiently loud to be audible outside the patient (e.g., using a stethoscope, or the unaided ear, applied to the surface of the patient's body). In some embodiments, the elongated tube 210 can define a whistle lumen 215 in fluid communication with the whistle feature. The whistle feature can define an opening such that fluid can flow from the whistle lumen 215, into an interior of the whistle feature, and out of the opening, producing the whistle sound audible outside the patient. In some embodiments, the opening can be defined in or aligned with an opening in a sidewall of the elongated tube 210. In some embodiments, the opening can be defined within the whistle feature at an intersection between an entrance passageway, an interior chamber, and an exit passageway of the whistle feature (e.g., in a configuration similar to a Storm® whistle, or any of the embodiments of similar whistles disclosed in U.S. Pat. No. 5,564,360, entitled “All Weather Safety Whistle and Sound Generator” (“the '360 Patent”), the disclosure of which is incorporated by reference herein. In some embodiments, the whistle feature can define an interior space in fluid communication with the whistle lumen 215, a closed distal end, and a projection (e.g., a bump) extending from an inner surface of the whistle feature toward the whistle opening. The whistle feature can be disposed at a distal end 216 of the whistle lumen 215. Thus, fluid can flow from the whistle lumen 215 into the open end of the whistle, flow into the interior space, accumulate beyond the projection in the interior space, and then flow out of the opening, producing a whistle sound resulting from interaction with the whistle feature. In some embodiments, the position indicator 260 can be formed similarly to a Storm® whistle (or other whistle disclosed in the '360 Patent) having an umbrella configured to define a chamber. In some embodiments, the position indicator 260 and/or a whistle lumen 215 can define a distal opening (e.g., through the first end 211 of the elongated tube 210) such that fluid pushed through the whistle lumen 215 and the position indicator 260 (e.g., the whistle feature) can travel through an exit passageway of the whistle feature and pass out the first end 211 of the elongated tube 210. In some embodiments, the whistle feature can be releasably couplable to the elongated tube 210.

In some embodiments, the system 200 can include an inflatable member 230 disposed on the elongated tube 210 and defining an interior in fluid communication with the whistle lumen 215 and an interior of the position indicator 260 (e.g., the whistle feature) to provide a gaseous space. The inflatable member 230 can be disposed such that the inflatable member 230 surrounds the position indicator 260 or a portion of the position indicator 260 defining an opening (e.g., a whistle opening) aligned with an opening of the elongated tube 210. In some embodiments, the inflatable member 230 can be inflated by gaseous fluid introduced into the whistle lumen 215 and through the position indicator 260. In some embodiments, the inflatable member 230 can surround a portion of the elongated tube 210. In some embodiments, the inflatable member 230 can be configured such that the inflatable member 230 is configured to transition between an unexpanded and an expanded configuration, and the inflatable member 230 has an outer diameter or maximum dimension perpendicular to a central axis of the elongated member 210 in the expanded configuration such that the inflatable member 230 cannot fully expand to the expanded configuration in a patient's trachea, lungs, esophagus, and/or jejunum, but can fully expand to the expanded configuration in a patient's stomach. In some embodiments, the inflatable member 230 is sized such that the inflatable member 230 cannot inflate to a sufficiently large volume (e.g., the expanded configuration) for the whistle feature to produce a sound, a sufficiently loud sound, or a sound of a sufficient duration in a patient's trachea, lungs, esophagus, and/or jejunum, but can inflate to a sufficiently large volume (e.g., the expanded configuration) for the whistle feature to produce a sufficiently loud sound (i.e., sufficient decibels) and/or a sound of a sufficiently long duration when disposed in the patient's stomach, to provide the desired indication. Thus, if the inflatable member 230 is disposed in a non-target location, such as a patient's trachea, lungs, esophagus, and/or jejunum, the user can determine that the inflatable member 230 is in the non-target location rather than in a target location (e.g., the stomach) because of a lack of audible indication or a change in the audible indication produced by the whistle feature when the inflatable member 230 is in the non-target location (due to the inflatable member 230 being prevented from inflating to the expanded configuration by the geometry of the non-target location).

In some embodiments, the position indicator 260 can be electronic. The position indicator 260 can include a sensor configured to sense a characteristic of fluid in contact with the position indicator 260 and generate an indication based on a presence of the sensed characteristic. In some embodiments, the characteristic can be the presence of liquid fluid. In some embodiments, the characteristic can be an acidity of fluid contacting the sensor being within a particular range (e.g., the pH range associated with stomach acid). In some embodiments, the position indicator 260 can generate an indication in response to a signal transmitted to the position indicator 260, e.g., generated by a user. In some embodiments, the sensor can include a noise-generating component such as a speaker to generate the notification, which can include a sound like a buzzer or other alarm. In some embodiments, the sensor can include a transmitter and be configured to communicate a status of the sensor (e.g., a sensed characteristic or a determination based on the sensed characteristic) to a remote compute device such as a smartphone via, e.g., Bluetooth®.

In some embodiments, the second end 213 of the elongated tube 210 can be coupled to and/or can include one or more pumps and/or sources of fluid. For example, a source of feeding fluid and/or an associated pump can be coupled to the lumen 212 to provide feeding fluid to the lumen 212. As shown in FIG. 2, a fluid and/or vacuum source 235 can be optionally included in the system 200 and can be fluidically coupled to the whistle lumen 215. In some embodiments, the fluid and/or vacuum source 235 can define a reservoir of gaseous fluid and can be manipulated (e.g., squeezed or pressed by a healthcare provider) to urge the gaseous fluid into the whistle lumen 215 and to the position indicator 260 including a whistle feature with a sufficient pressure and/or flow rate such that the position indicator 260 can generate an audible whistle. In some embodiments, the fluid and/or vacuum source 235 can include a flexible bulb defining a reservoir sealed to the elongated tube 210. The flexible bulb can be squeezed to push gaseous fluid (e.g., a bolus of gaseous fluid) into the whistle lumen 215 and can be biased toward an expanded configuration such that when the flexible bulb is released gaseous fluid (e.g., the gaseous fluid pushed out of the flexible bulb) is drawn into the flexible bulb. In some embodiments, the flexible bulb, the whistle lumen 215, the position indicator 260, and the inflatable member 230 can form a closed fluid system such that gaseous fluid can, upon squeezing the flexible bulb, be pushed from the flexible bulb into the whistle lumen 215, through the position indicator 260 to produce a whistle, and into the inflatable member 230, and then, upon releasing the flexible bulb, the flexible bulb expands to draw the fluid from the inflatable member 230, through the position indicator 260, through the whistle lumen 215, and into the flexible bulb. In some embodiments, rather than a flexible bulb, the fluid and/or vacuum source 235 can include a syringe defining a reservoir and couplable to the whistle lumen 215 such that the syringe can be manipulated to push fluid (e.g., a bolus) to the position indicator 260 via the whistle lumen 215 and draw fluid from the position indicator 260 via the whistle lumen 215. In some embodiments, due to the fluid and/or vacuum source 235, the whistle lumen 215, the position indicator 260, and the inflatable member 230 forming a closed fluid system, if the inflatable member 230 is disposed in a location (e.g., a trachea, esophagus, lung, or small bowel) that restrains the inflatable member 230 and does not allow the inflatable member 230 to fully expand to an expanded configuration, the fluid and/or vacuum source 235 will resist being fully pushed or depressed, providing feedback in the form of a tactile indication to the user that the inflatable member 230, and thus the first end 211 of the elongated member 210, is in the wrong location within the patient. In some embodiments, the user can determine that the inflatable member 230 is in the wrong location based on the resistance to pushing or squeezing the fluid source 235 (e.g., the flexible bulb or syringe) (e.g., based on being above a threshold resistance or based on a change in resistance partially through a push, squeeze, or depression) and/or based on a volume remaining in the fluid source 235 when the resistance increases or significantly increases (e.g., compared to a volume of fluid remaining in the fluid source 235 when the inflatable member 230 is unconstrained and in the expanded configuration). In response to the resistance or increase in resistance sensed by the user, the user can cease applying pressure to the fluid source 235 and manipulate (e.g., translate) the elongated tube 210 to move the inflatable member 230 and the first end 211 to a new location to test. In some embodiments, the system 200 can optionally include a pressure release valve fluidically coupled to the inflatable member 230 and the whistle lumen 215 and configured to release fluid when a fluid pressure within the inflatable member 230 and the whistle lumen 215 is above a threshold fluid pressure to prevent damage to the system 200 (e.g., to prevent bursting of the inflatable member 230). In some embodiments, the inflatable member 230 can be the position indicator 260, and the system 200 may not include an auditory feedback producing component (e.g., a whistle).

In some embodiments, the elongated tube 210 can define any suitable number of additional lumens. For example, as shown in FIG. 2, the elongated tube 210 can define an additional lumen 217. The lumen 217 can be in fluid communication with an opening 218 disposed in the first end 211 of the elongated tube 210. The lumen 217 can be used for fluid delivery and/or suction. In some embodiments, the lumen 212 can be the only lumen and/or can be in fluid communication with the position indicator 260 such that fluid traveling through the lumen 212 at a particular flow rate and/or pressure can activate the position indicator 260 (e.g., the whistle feature). For example, the lumen 212 can be used to provide fluid including nutrient and/or medication to a stomach of a patient during a first time period, then the lumen 212 can be cleared by pushing air through the lumen 212, and then the lumen 212 can be used as a whistle lumen to check the position of the position indicator 260. Additionally, although the stomach is described herein as the intended location of the first end 211 of the elongated tube 210, in some embodiments, the first end 211 of the elongated tube 210 can be configured to be placed distally of the stomach and disposed in the small bowel of the patient. In such embodiments, the elongated tube 210 can be longer and the inflatable member 230, the elongated tube 210, and the position indicator 260 (e.g., whistle feature) can each have a smaller maximum diameter than if the first end 211 is intended for placement in the stomach.

In some embodiments, the elongated tube 210 can optionally include one or more magnetic members 220 disposed near at or near the first end 211 of the elongated tube 210. In some embodiments, the one or more magnetic members 220 can be disposed distal to none, some, or all of the openings 214 (e.g., sidewall fenestrations) in fluid communication with the lumen 212. In some embodiments, the one or more magnetic members 220 can be disposed within the elongated tube 210. For example, the elongated tube 210 can include an end cap coupled to the first end 211, and one or more magnetic members 220 can be enclosed within a cavity or lumen of the elongated tube 210 bounded by the end cap. In some embodiments, the one or more magnetic members 220 can be formed in any suitable shape or size and can include any suitable number of magnetic members. For example, in some embodiments, the one or more magnetic members 220 can include one or more spherical magnetic components (e.g., one, two, three, four, or more) disposed within the elongated tube 210 and/or an end cap coupled to the first end 211 of the elongated tube 210 (e.g., in a line).

In some embodiments, the one or more magnetic members 220 can be formed of any suitable type of magnet. For example, the one or more magnetic members 220 can include a permanent magnet, such as a rare earth magnet (such as neodymium iron boron (NdFeB) or samarium cobalt (SmCo)), an aluminum nickel cobalt (AlNiCo) magnet, a ceramic/ferrite magnet, and/or any other suitable permanent magnet. In some embodiments, the one or more magnetic members 220 can include a temporary magnet. In some embodiments, the one or more magnetic members 220 can be an electromagnet, such as a solenoid (with or without a solid core). In some embodiments, the one or more magnetic members 220 can generate a magnetic field having an orientation (i.e., north (N) and south (S) poles). In other embodiments, the magnetic feature 220 can be formed of a ferromagnetic material that is not magnetized, i.e., does not generate its own magnetic field, but can be affected by an externally-applied magnetic field. For example, the one or more magnetic members 220 can be formed of iron, and application of an external magnetic field can attract the iron toward the source of the field, applying a force to the one or more magnetic members 220.

A position of the first end 211 within a cavity or passageway of a patient (e.g., a trachea, esophagus, and/or stomach) can be controlled by an external magnetic member disposed on a surface of a patient and configured to magnetically interact with the one or more magnetic members 220. An external magnetic member can be configured to be disposed on the surface of a patient's skin and can apply a magnetic field to at least a portion of a patient's body. The magnetic field of the external magnetic member can interact with the magnetic field generated by the one or more magnetic members 220 through various body tissue and organs disposed between the external magnetic member on the surface of the patient's skin the one or more magnetic members 220 within the patient's body and across any suitable distance (e.g., about 1 cm, 5 cm, 10 cm, about 15 cm, and/or about 20 cm). The interaction between the magnetic field generated by the external magnetic member and the magnetic field generated by the one or more magnetic members 220 can produce a magnetic force that urges the position of the one or more magnetic members 220 in a direction toward or away from the external magnetic member, and thus also urges the first end 211 of the elongated tube 210 relative to the external magnetic member. For example, the external magnetic member can be disposed on a front or back surface of a patient's neck to urge the first end 211 of the elongated tube 210 toward the back of the patient's throat and down the esophagus rather than the trachea of the patient as the first end 211 is translated through the patient. In some embodiments, the external magnetic member and/or the magnetic member(s) 220 can be the same or similar in structure and/or function to any of the external magnetic members or magnetic members disclosed in International Application No. PCT/US2019/030902, filed May 6, 2019, entitled “Systems, Apparatus, and Methods for Delivering a Medical Device Beyond a Bifurcation in a Body Lumen,” published as International Publication Number WO 2019/213657, which is incorporated by reference herein in its entirety.

In use, the first end 211 of the elongated tube 210 can be inserted through a nasal or oral orifice of the patient and translated relative to the orifice. The first end 211 can be guided to and through the esophagus of the patient using any suitable method. For example, the first end 211 can be navigated beyond an internal junction of the patient to avoid the trachea and instead travel into the esophagus with the aid of an external magnetic assembly applied to the neck of the patient to urge the magnetic member(s) 220 posteriorly as the elongated tube 210 is translated. In some embodiments, the first end 211 can be translated into the patient blindly or with the aid of an endoscope. With the first end 211 of the elongated tube 210 disposed in a cavity or passageway of the patient, the location of the first end 211 can be verified to confirm that the first end 211 is disposed in a target cavity or location (e.g., the stomach), rather than in an unintended location such as the trachea, lungs, esophagus, and/or small bowel.

To identify the location of the first end 211 of the elongated tube 210 within the patient, the position indicator 260 can be used to provide any of the indications described above. For example, fluid flow through a lumen of the elongated tube 210 (e.g., the whistle lumen 215) can be produced such that the fluid flow interacts with a whistle feature of the position indicator 260. If the interaction between the fluid flow and the whistle feature produces a whistle sound indication audible outside of the patient to the user (e.g., a healthcare provider), the user can determine which portion of the patient's body is the source of the whistle sound indication and thus can determine whether that portion is associated with the patient's stomach, small bowel, esophagus, etc. If the whistle sound indication is coming from the patient's stomach, the user can then provide liquid fluid including nutrient and/or medication to the patient's stomach via the elongated tube 210. If the whistle sound indication is coming from a location other than the patient's stomach or no whistle sound indication is audible to the user (e.g., due to the inflatable member 230 being restrained from sufficiently expanding to provide a gaseous space for the whistle feature), the user can withdraw the elongated tube 210 and/or reposition the elongated tube 210 and recheck the location of the first end 211.

In some embodiments, instead of, or in addition to, listening for an audible indication, the fluid and/or vacuum source 235 can, in combination with the position indicator 260, which may include or be formed as the inflatable member 230, provide a tactile indicator to the user that the first end 211 of the elongated tube 210 is not disposed in the target cavity. For example, as described above, the fluid and/or vacuum source 235 can be manipulable to urge a bolus of gaseous fluid into a lumen of the elongated tube (e.g., the whistle lumen), into interaction with the position indicator, and into the interior of the inflatable member 230 to expand the inflatable member 230. If the user senses increased resistance to further manipulation of the fluid and/or vacuum source 235 before delivering the entire bolus (e.g., the fluid and/or vacuum source 235 becomes harder to squeeze), the increased resistance can be a tactile indicator that the inflatable member 230 is not disposed in the target cavity (e.g., the stomach) where the inflatable member 230 would be able to fully expand, and is instead located in an unintended location, such as the esophagus or trachea. If the tactile indication suggests that the inflatable member 230 is not disposed in the target cavity (e.g., due to the inflatable member 230 being restrained from fully expanding), the user can withdraw the elongated tube 210 and/or reposition the elongated tube 210 and recheck the location of the first end 211. If the user does not perceive a tactile indication that the inflatable member 230 is restrained from fully expanding (e.g., the user perceives that the full bolus is delivered without excessive force by the user, which can be a positive tactile indication), the user can provide liquid fluid through the lumen 212 of the elongated tube 210 to the target cavity (e.g., the stomach).

In some embodiments, after initially confirming that the first end 211 is disposed in the target cavity via the above steps or another suitable method (e.g., via X-ray imaging), any of the above steps can be used to confirm that the first end 211 of the elongated tube 210 has not migrated unsafely relative to the target cavity after a period of time. For example, the above steps can be used to confirm that the first end 211 of the elongated tube 210 has not migrated into the esophagus or small bowel. In some embodiments, the location of the first end 211 of the elongated tube 210 can be checked, for example, every four hours or before every administration of fluid via the elongated tube 210.

In some embodiments, as an alternative to, or in addition to, the position indicator 260 and the inflatable member 230 described above, the system 200 can include a first securing inflatable member and a second securing inflatable member (also referred to as balloons or cuffs) coupled to the elongated tube 210 and disposed proximally of the one or more openings 214 (e.g., proximal of any sidewall fenestrations). The first securing inflatable member and the second securing inflatable member can be coupled to the elongated member 210 such that the first securing inflatable member is disposed distally of the second securing inflatable member. In some embodiments, the first securing inflatable member and the second securing inflatable member can be coaxially arranged relative to each other and the elongated member 210. In some embodiments, no openings in a sidewall of the elongated member 210 are defined between the first securing inflatable member and the second securing inflatable member. In some embodiments, the first securing inflatable member and the second securing inflatable member can be fluidically isolated from each other and can each be fluidically coupled to respective inflation lumens. In some embodiments, rather than two distinct securing inflatable members, the system 200 can include a single securing inflatable member having a smaller diameter midportion separating the inflatable member into two separately inflatable portions (e.g., via a belt coupled to the midportion).

In use, after disposing the first end 211 of the elongated tube 210 in the stomach of the patient and with the elongated tube 210 extending through the patient's esophagus and out of the patient's nasal or oral orifice, the first securing inflatable member, which is disposed distally of the second securing inflatable member, can be expanded from an unexpanded to an expanded configuration (e.g., inflated). The elongated tube 210 can then be translated (e.g., pulled) proximally such that the first securing inflatable member is pulled against the esophageal sphincter of the patient and the second securing inflatable member is disposed within the esophagus of the patient. The second securing inflatable member can then be expanded from an unexpanded to an expanded configuration (e.g., inflated). Using an appropriate imaging device, such as an ultrasound probe, the system 200 can be visualized to confirm that the second securing inflatable member is disposed within the esophagus. Thus, the first securing inflatable member and the second securing inflatable member can be used to confirm the position of the first end 211 of the elongated tube 210 and to secure the first end 211 relative to the esophageal sphincter. Nutrient fluids and/or medication can then be delivered through the lumen 212 into the stomach.

In some embodiments, after disposing the first end 211 of the elongated tube 210 in the stomach of the patient and with the elongated tube 210 extending through the patient's esophagus and out of the patient's nasal or oral orifice, the second securing inflatable member can be expanded from an unexpanded to an expanded configuration (e.g., inflated). The first end 211 of the elongated tube 210 can then be urged distally into the small bowel (e.g., the duodenum) until the second securing inflatable member contacts the pylorus and the first securing inflatable member is disposed within the small bowel. The first securing inflatable member can then be expanded from an unexpanded to an expanded configuration (e.g., inflated) within the small bowel. Using an appropriate imaging device, such as an ultrasound probe, the system 200 can be visualized to confirm if the second securing inflatable member is in the small bowel. Thus, the first securing inflatable member and the second securing inflatable member can be used to confirm the position of the first end 211 of the elongated tube 210 and to secure the first end 211 relative to the pylorus. Nutrient fluids and/or medication can then be delivered through the lumen 212 into the small bowel. In some embodiments, the elongated tube 210 can define fenestrations proximal of the second securing inflatable member in fluidic communication with fenestrations distal of the first securing inflatable member.

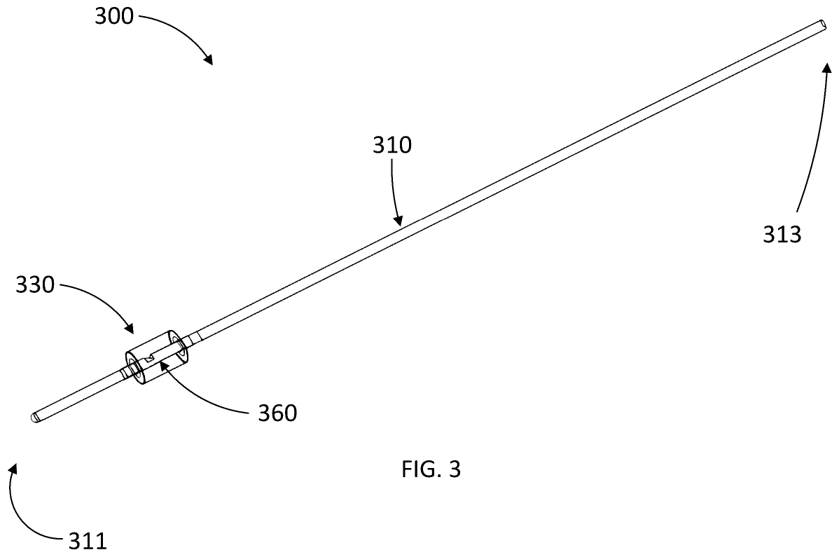

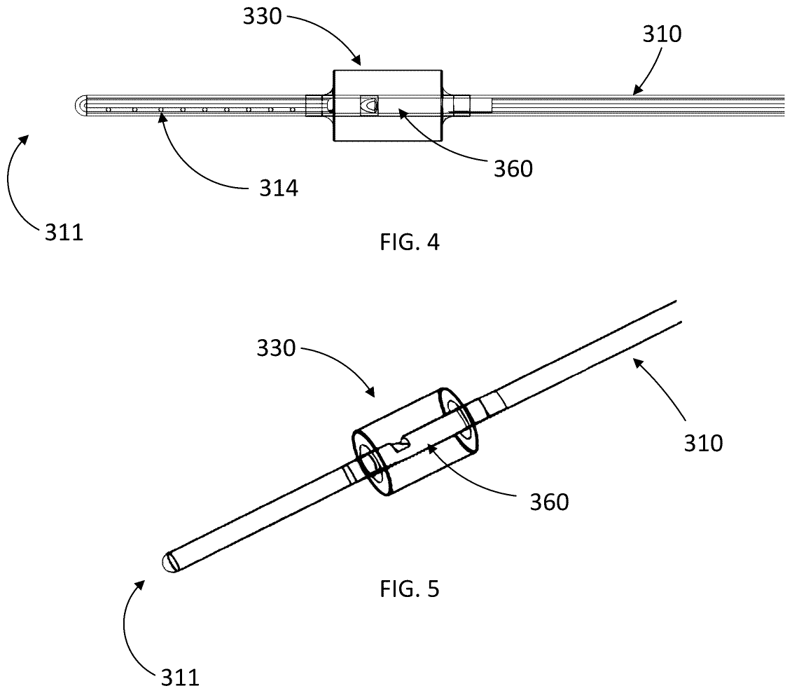

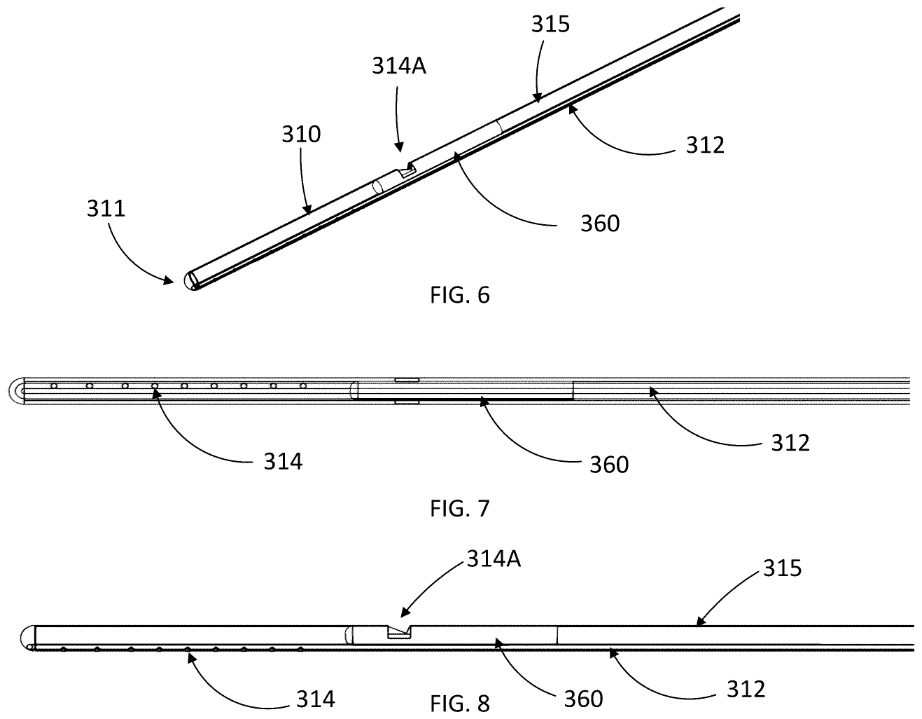

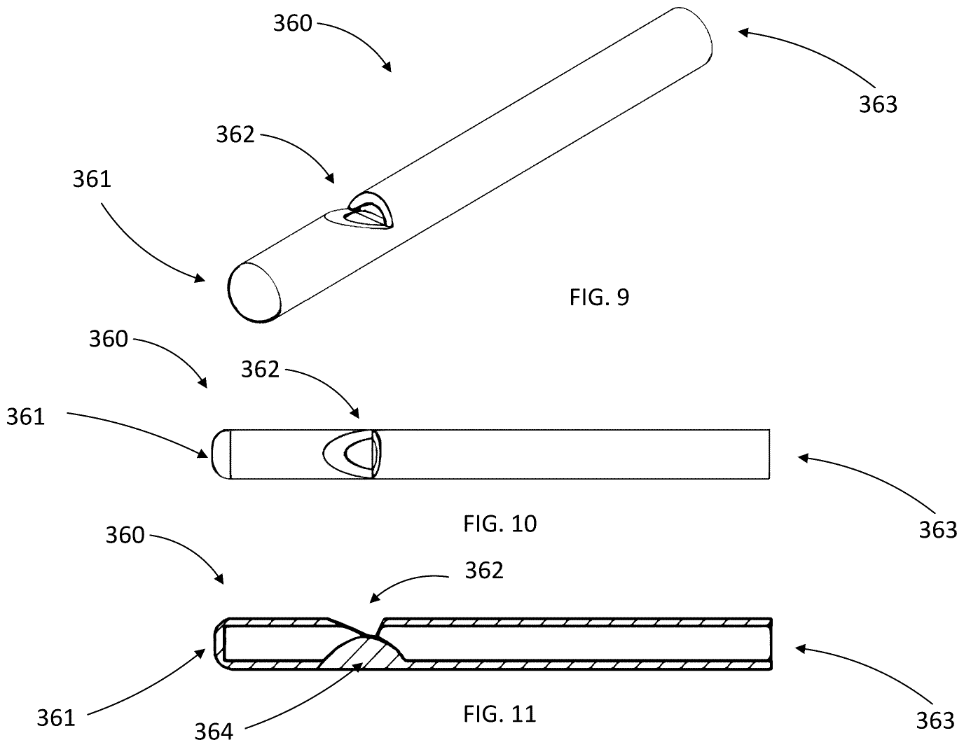

FIGS. 3-11 are various views of a system 300, which can be the same or similar in structure and/or function to any of the systems described herein, such as the system 200. As shown in FIG. 3, which is a perspective view of the system 300, the system 300 includes an elongated tube 310 having a first end 311 and a second end 313, a position indicator 360 associated with the first end 311 of the elongated tube 310, and an inflatable member 330 coupled to the position indicator 360. The elongated tube 310, the position indicator 360, and the inflatable member 330 can be the same or similar in structure and/or function to any of the elongated tubes, position indicators, and inflatable members shown and described herein. FIGS. 4 and 5 are a top view and a perspective view, respectively, of a distal portion of the system 300. FIGS. 6, 7, and 8 are a perspective view, a bottom view, and a side view, respectively, of the distal portion of the system 300 with the inflatable member 330 not shown. FIGS. 9, 10, and 11 are a perspective view, a top view, and a cross-sectional view, respectively, of the position indicator 360.

The elongated tube 310 defines a feeding lumen 312 and a whistle lumen 315. The feeding lumen 312 extends from the second end 313 to a location near the first end 311 of the elongated tube 310. The elongated tube 310 can define a set of fenestrations 314 (also referred to as “openings”) defined in a sidewall of the elongated tube 310 in fluid communication with the lumen 312 such that, when the first end 311 of the elongated tube 310 is disposed in a cavity of a patient (e.g., a stomach) and the second end 313 of the elongated tube 310 is disposed outside of the patient, fluid can be delivered from the second end 313 of the elongated tube 310 into the cavity via the feeding lumen 312 and the set of fenestrations 314. As shown, the first end 311 of the elongated tube 310 is a closed end such that fluid communication between the feeding lumen 312 and a body cavity (e.g., the stomach) within which the first end 311 is disposed is only via the set of fenestrations 314 in the sidewall.

As shown, the position indicator 360 is disposed sufficiently close to the first end 311 such that, when the first end 311 of the elongated tube 310 is properly positioned in a stomach of the patient and the second end 313 is disposed outside of the patient (e.g., beyond the patient's nose or mouth), the position indicator 360 is also positioned in the stomach of the patient. The position indicator 360 is disposed proximal to the set of fenestrations 314 and within the whistle lumen 315. The position indicator 360 can be formed as or include a whistle feature in fluid communication with the whistle lumen 315 such that gaseous fluid (e.g., air, nitrogen, carbon dioxide, and/or oxygen) flowing through the whistle lumen 315 can reach the position indicator 360 and cause the position indicator 360 to generate a whistle sound indication audible outside the patient.

As shown in FIGS. 9-11, the position indicator 360 can include an open end 363 and a closed end 361 and can define an opening 362 such that fluid can flow from the whistle lumen 315, through the open end 363 into an interior space defined by the position indicator 360, and out of the opening 362 to produce a whistle sound audible outside the patient. The opening 362 can be aligned with an opening 314A defined in a sidewall of the elongated tube 310. The position indicator 360 can also include a projection 364 (e.g., a bump) extending from an inner surface of the position indicator 360 toward the whistle opening 362. Thus, when a bolus of gaseous fluid is delivered to the whistle lumen 315, fluid can flow from the whistle lumen 315 into the open end 363 of the position indicator 360, flow into the interior space, accumulate in the portion of the interior space between the projection 364 and the closed end 361, and then flow out of the opening 362, producing a whistle sound resulting from interaction with the position indicator 360. In some embodiments, if the opening 362 is obstructed (e.g., by body tissue, liquid fluid, or a sidewall portion of the optional inflatable member 330 in an unexpanded or not fully expanded configuration), the position indicator 360 will be prevented from producing the audible whistle sound indication or producing a whistle sound reaching a threshold decibel level.

The inflatable member 330 can define an interior in fluid communication with the interior space of the position indicator 360 and the whistle lumen 315. The inflatable member 330 can be configured to transition between an unexpanded configuration and the expanded configuration shown in FIGS. 3-5 as a result of gaseous fluid being provided to the position indicator 360 via the whistle lumen 315 and passing from the opening 362 of the position indicator 360 into the interior space of the inflatable member 330. As shown in FIGS. 3-5, the inflatable member can fully surround a portion of the elongated member 310 in the expanded configuration. The inflatable member 330 can be configured to provide a gaseous space within a stomach of the patient fluidically coupled to the opening 362 such that the position indicator 360 produces an audible indication (e.g., a whistle sound) that is audible outside of the patient. If the inflatable member 330 is prevented from sufficiently expanding to or toward the expanded configuration, insufficient gaseous space will be available for the position indicator 360 to produce an audible indication that can be heard or heard sufficiently clearly outside the patient.

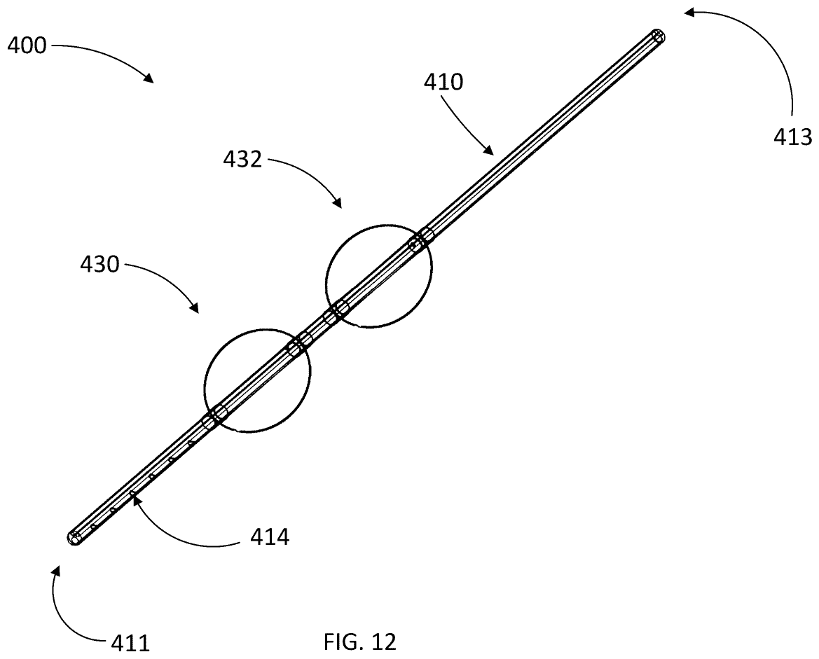

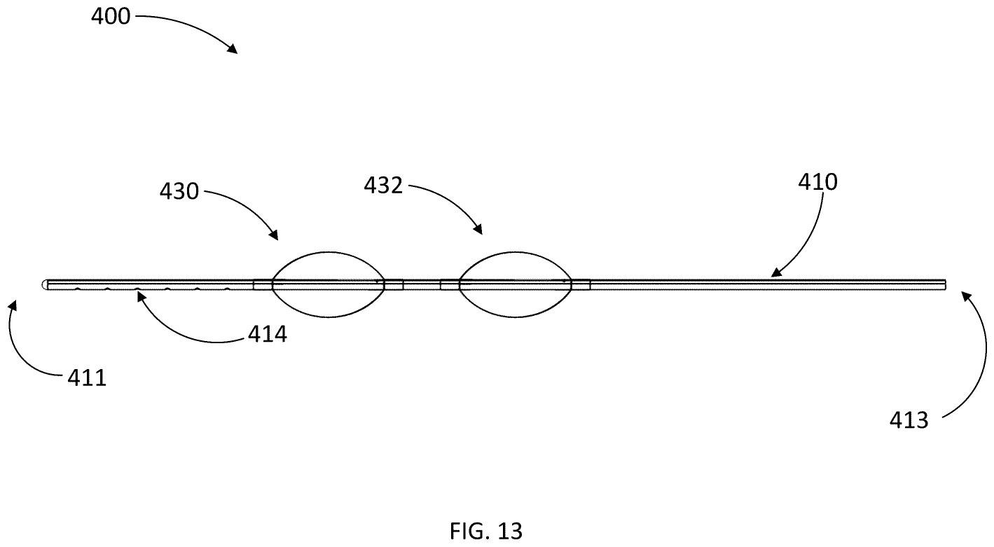

In some embodiments, as an alternative to or in addition to being coupled to one or more of the position indicators described herein, an elongated tube (e.g., a nasogastric tube) can be coupled to one or more inflatable members configured to fix the position of the elongated tube relative to a patient cavity and/or passageway. For example, FIGS. 12 and 13 are a perspective view and a side view of a system 400. The system 400 can be the same or similar in structure and/or function to any of the systems described herein. The system 400 includes an elongated tube 410 having a first end 411 and a second end 413, a first securing inflatable member 430, and a second securing inflatable member 432. The elongated tube 410, the first securing inflatable member 430, and the second securing inflatable member 432 can be the same or similar in structure and/or function to any of the elongated tubes and inflatable members shown and described herein.

The elongated tube 410 can define a feeding lumen and a set of fenestrations 414 (also referred to as “openings”) defined in a sidewall of the elongated tube 410 in fluid communication with the lumen such that, when the first end 411 of the elongated tube 410 is disposed in a cavity of a patient (e.g., a stomach) and the second end 413 of the elongated tube 410 is disposed outside of the patient, fluid can be delivered from the second end 413 of the elongated tube 410 into the cavity via the feeding lumen and the set of fenestrations 414.

The first securing inflatable member 430 and the second securing inflatable member 432 are coupled to the elongated tube 410 and disposed proximally of the set of fenestrations 414. The first securing inflatable member 430 and the second securing inflatable member 432 can be coupled to the elongated member 410 such that the first securing inflatable member 430 is disposed distally of the second securing inflatable member 432. As shown, the first securing inflatable member 430 and the second securing inflatable member 432 can be coaxially arranged relative to each other and the elongated member 410, and no openings are defined in the portion of the sidewall of the elongated member 410 extending between the first securing inflatable member 430 and the second securing inflatable member 432. The first securing inflatable member 430 and the second securing inflatable member 432 are fluidically isolated from each other and separately inflatable (e.g., via separate inflation lumens defined by the elongated member 410). In some embodiments, one or both of the first securing inflatable member 430 and the second securing inflatable member 432 can be coupled to, associated with, or include any of the position indicators described herein, such as, for example, a whistle feature. In some embodiments, the elongated tube 410 is not coupled to or associated with an audible indication generating position indicator.

In use, after disposing the first end 411 of the elongated tube 410 in the stomach of the patient and with the elongated tube 410 extending through the patient's esophagus and out of the patient's nasal or oral orifice, the first securing inflatable member 430, which is disposed distally of the second securing inflatable member 432, can be expanded from an unexpanded to an expanded configuration (e.g., inflated). The elongated tube 410 can then be translated (e.g., pulled) proximally such that the first securing inflatable member 430 is pulled against the esophageal sphincter of the patient and the second securing inflatable member 432 is disposed within the esophagus of the patient. The second securing inflatable member 432 can then be expanded from an unexpanded to an expanded configuration (e.g., inflated). Using an ultrasound probe, the system 400 can be visualized to confirm that the second securing inflatable member 432 is disposed within the esophagus. Thus, the first securing inflatable member 430 and the second securing inflatable member 432 can be used to confirm the position of the first end 411 of the elongated tube 410 and to secure the first end 411 relative to the esophageal sphincter. Nutrient fluids and/or medication can then be delivered through the lumen into the stomach.

In some embodiments, after disposing the first end 411 of the elongated tube 410 in the stomach of the patient and with the elongated tube 410 extending through the patient's esophagus and out of the patient's nasal or oral orifice, the second securing inflatable member 432 can be expanded from an unexpanded to an expanded configuration (e.g., inflated). The first end 411 of the elongated tube 410 can then be urged distally into the small bowel (e.g., the duodenum) until the second securing inflatable member 432 contacts the pylorus and the first securing inflatable member 430 is disposed within the small bowel. The first securing inflatable member 430 can then be expanded from an unexpanded to an expanded configuration (e.g., inflated) within the small bowel. Using an ultrasound probe, the system 400 can be visualized to confirm if the second securing inflatable member 432 is in the small bowel. Thus, the first securing inflatable member 430 and the second securing inflatable member 432 can be used to confirm the position of the first end 411 of the elongated tube 410 and to secure the first end 411 relative to the pylorus. Nutrient fluids and/or medication can then be delivered through the lumen 412 into the small bowel. In some embodiments, the elongated tube 410 can define fenestrations proximal of the second securing inflatable member 432 in fluidic communication with fenestrations distal of the first securing inflatable member 430.

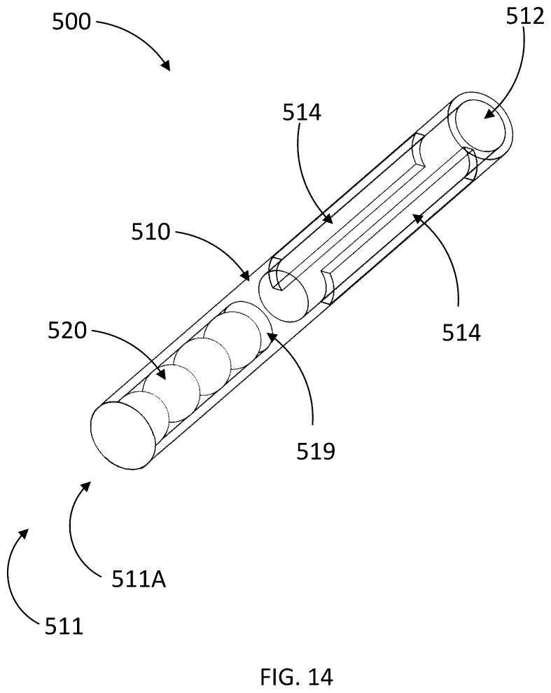

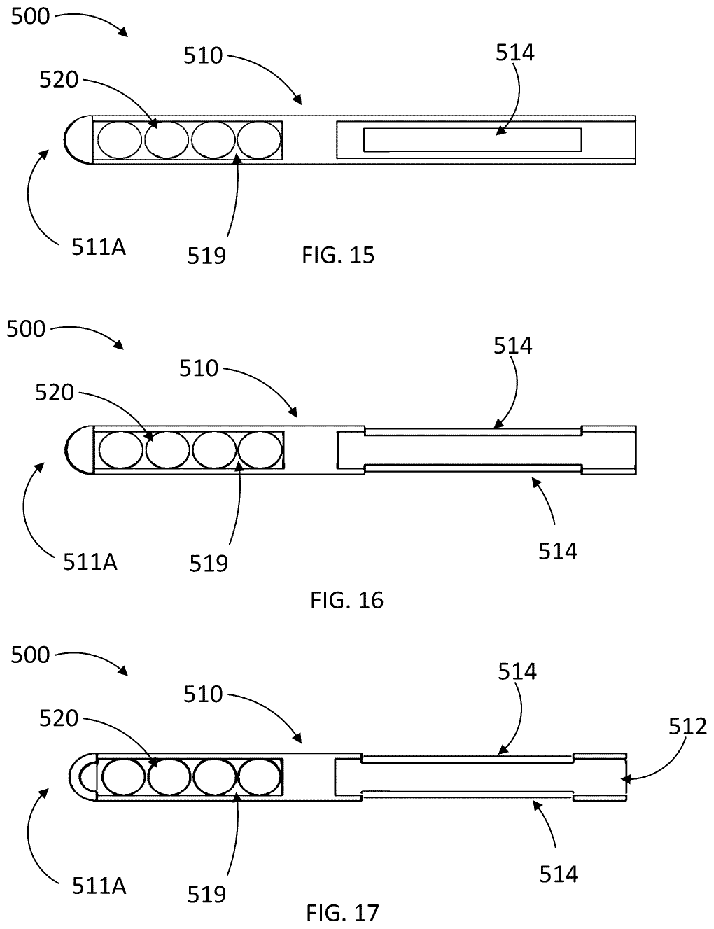

FIGS. 14-17 are various views of a portion of a distal portion of a system 500. FIG. 14 is a perspective view of the distal portion of the system 500. FIGS. 15 and 16 are a side view and a top view, respectively, of the distal portion of the system 500. FIG. 17 is a cross-sectional view of the distal portion of the system 500. The system 500 can be the same or similar in structure and/or function to any of the systems described herein. For example, the system 500 includes an elongated tube 510 defining a lumen 512 and fenestrations 514 in fluid communication with the lumen 512 near a first end of the elongated tube 510. The elongated tube 510 also defines a cavity 519 bounded by the elongated tube 510 and a cap 511A disposed on the distal end of the elongated tube 511. A set of magnetic members 520 is disposed in the cavity 519. The set of magnetic members 520 can be the same or similar in structure and/or function to any of the magnetic members described herein, such as the magnetic member(s) 220. Although four magnetic members 520 are shown, any suitable number of magnetic members 520 can be included in the set. Although the magnetic members 520 are shown as being spherical, the magnetic members 520 can have any suitable shape. The set of magnetic members 520 can be configured to magnetically interact with an external magnetic assembly such that the external magnetic assembly can be used to urge the magnetic members 520, and thus the first end 511 of the system 500, toward or away from the external magnetic assembly such that the first end 511 can be navigated through the patient (e.g., into the esophagus and into the stomach of the patient). After delivering the first end 511 to the stomach of the patient, fluid (nutrients and/or medication) can be delivered to the stomach via the lumen 512 and the fenestrations 514.

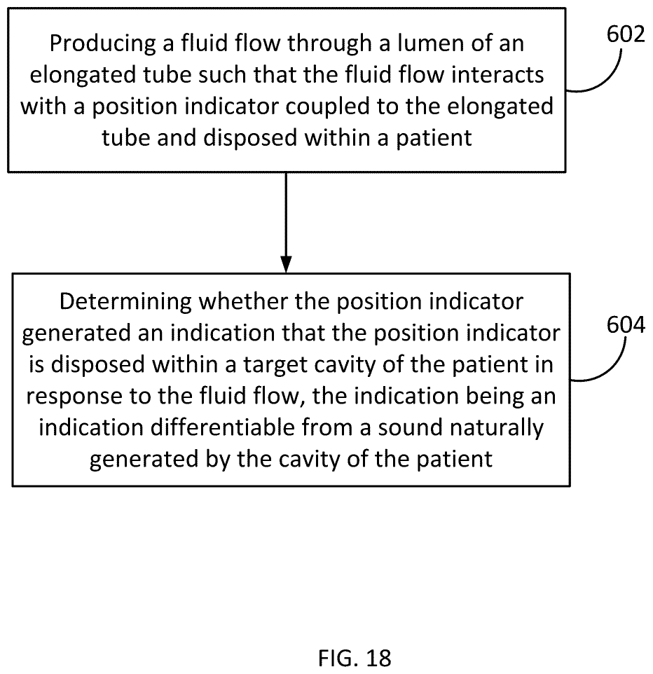

FIG. 18 is a flow chart illustrating a method 600 of using any of the systems and/or assemblies described herein. The method 600 includes producing, at 602, a fluid flow through a lumen of an elongated tube such that the fluid flow interacts with a position indicator coupled to the elongated tube and disposed within a patient. The method 600 further includes determining, at 604, whether the position indicator generated an indication that the position indicator is disposed within a target cavity of the patient in response to the fluid flow, the indication being an indication differentiable from a sound naturally generated by the cavity of the patient. In some embodiments, the determining includes listening for whether an audible indication is emitted from a portion of a body of the patient associated with the target cavity. If no audible indication is emitted or no audible indication is emitted from the portion of the body of the patient associated with the target cavity, the position indicator can be determined to be not disposed in the target cavity. For example, no audible indication may be emitted because the position indicator is unable to generate the audible indication due to being disposed in a non-target cavity or passageway, such as because an inflatable member of the position indicator is unable to sufficiently inflate such that the audible indication can be produced. In some embodiments, no audible indication may be emitted from the portion of the body of the patient associated with the target cavity because the position indicator is disposed in a non-target cavity or passageway and an audible indication is emitted from a portion of the body of the patient not associated with the target cavity. In some embodiments, the position indicator can include an inflatable member, and the producing can include applying pressure to a source of the fluid flow. The source of the fluid flow can be in fluid communication with the inflatable member via the lumen. The determining can include sensing a resistance of the source of the fluid flow to the applied pressure. For example, a resistance above a threshold resistance can be sensed (e.g., based on a resistance of a syringe plunger or a flexible bulb of the fluid source from being pressed to urge fluid into the inflatable member, or based on a volume of the syringe or bulb of the fluid source when the resistance increases signifying that the inflatable member is resistant to receiving additional fluid from the fluid source that the inflatable member would not be resistant or would be less resistant to receiving if the inflatable member were unconstrained). The resistance can be associated with the inflatable member being disposed within a non-target cavity or passageway that is not sufficiently large (e.g., lacks a sufficiently large cross-section) such that the inflatable member can expand to a particular diameter or volume (e.g., associated with the inflatable member when unconstrained and inflated within the target cavity and/or sufficiently large such that the indication can be produced). In some embodiments, upon determining that the position indicator is disposed in the target cavity, liquid fluid can be provided through a feeding lumen of the elongated tube to the target cavity. In some embodiments, upon determining that the position indicator is not disposed in the target cavity, the elongated tube can be manipulated (e.g., translated) to relocate the position indicator (e.g., from a non-target cavity or passageway to the target cavity). In some embodiments, the determining that the position indicator is not disposed in the target cavity includes determining that the indication was not produced.

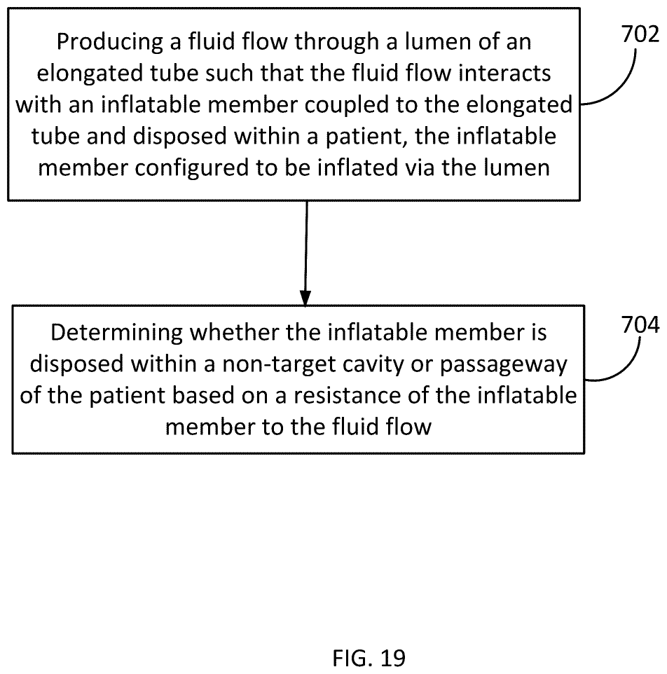

FIG. 19 is a flow chart illustrating a method 700 of using any of the systems and/or assemblies described herein. The method 700 includes producing, at 702, a fluid flow through a lumen of an elongated tube such that the fluid flow interacts with an inflatable member coupled to the elongated tube and disposed within a patient. The inflatable member is configured to be inflated via the lumen. The method 700 further includes determining, at 704, whether the inflatable member is disposed within a non-target cavity or passageway of the patient based on a resistance of the inflatable member to the fluid flow. The producing can optionally include applying pressure to a source of the fluid flow, the source being in fluid communication with the inflatable member via the lumen. The determining can optionally include sensing a resistance of the source of the fluid flow to the applied pressure. For example, a resistance above a threshold resistance can be sensed (e.g., based on a resistance of a syringe plunger or a flexible bulb of the fluid source from being pressed to urge fluid into the inflatable member, or based on a volume of the syringe or bulb of the fluid source when the resistance increases signifying that the inflatable member is resistant to receiving additional fluid from the fluid source that the inflatable member would not be resistant or would be less resistant to receiving if the inflatable member were unconstrained). In some embodiments, upon determining that the inflatable member is not disposed in the non-target cavity or passageway, liquid fluid can be provided through a feeding lumen of the elongated tube to a target cavity. In some embodiments, upon determining that the inflatable member is disposed in the non-target cavity or passageway, the elongated tube can be manipulated to relocate the inflatable member. In some embodiments, the inflatable member is included in a position indicator, and the method can further include determining whether the position indicator generated an indication that the position indicator is disposed within a target cavity of the patient in response to the fluid flow. For example, upon determining that the inflatable member is not disposed in the non-target cavity or passageway (e.g., based on a lack of resistance to inflation of the inflatable member and/or the volume of fluid delivered to the inflatable member), the user can determine whether the position indicator generates an indication that the position indicator is disposed within the target cavity. The indication can be an indication differentiable from a sound naturally generated by the cavity of the patient. In some embodiments, the indication can be an audible indication and the determining can include listening for whether the audible indication is emitted from a portion of a body of the patient associated with the target cavity. For example, the position indicator can include a whistle feature and the user can deliver fluid through a whistle lumen and listen for a whistle to determine if the inflatable member is properly inflated (and thus disposed in a sufficiently large cavity such as the target cavity) and/or to determine if the position indicator is in the target cavity based the source of the audible indication relative to the patient's body. Thus, in some embodiments, the placement of the distal end of an elongated tube can include a first step of confirming that an inflatable member of a position indicator is able to properly expand to an expanded configuration in a location, suggesting that the inflatable member is in a sufficiently large cavity that is likely to be the target cavity (e.g., the stomach) and not a non-target cavity or passageway (e.g., the esophagus, trachea, bronchi, or lungs), and a second step of confirming that an indication is able to be produced by the position indicator and/or that the indication is emitted from a portion of a patient's body associated with (e.g., aligned with) the target cavity (e.g., the stomach).

In some embodiments, rather than or in addition to including a position indicator, such as any of the position indicators described herein (e.g., position indicator 260), that is disposable within a patient and provides a user-perceptible indication from within the patient, a system, such as any of the systems described herein, can include or be coupled to a visual indicator configured to provide an indication that is visible to a user (e.g., an operator of the system such as a clinician) and that is associated with a position or location of a distal end of an elongated tube within a patient's body. In some embodiments, the visual indicator can be disposed external to the patient (e.g., can be coupled to a portion of the elongated tube, such as a proximal end, that remains external to the patient during an elongated tube distal end placement procedure). The visual indicator can be configured to be disposed in the line of sight of a user (e.g., a clinician) who is handling the system and facing the patient. In some embodiments, the visual indicator can be configured to provide one or more indications associated with the distal end of the elongated tube being properly and/or improperly disposed within a patient. For example, the visual indicator can be configured to provide an indication that the distal end of the elongated tube is disposed within a target location (e.g., within a body region having characteristics consistent with a target location, such as a region having a sufficiently large cross-sectional area or diameter) and/or to provide an indication that the distal end of the elongated tube is disposed in a non-target location (e.g., a region having characteristics inconsistent with the target location, such as a region having a smaller cross-sectional area or diameter than the target location). In some embodiments, the non-target location can be upstream or downstream of the target location.

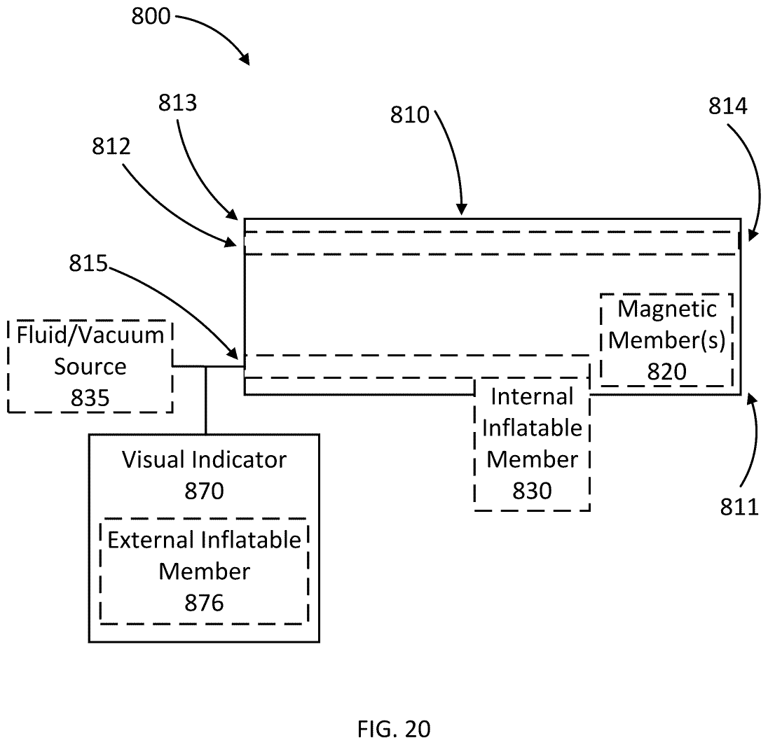

For example, FIG. 20 is a schematic illustration of a system 800. The system 800 can be the same or similar in structure and/or function to any of the systems described herein, such as the system 200 described above with respect to FIG. 2. The system 800 includes an elongated tube 810 and a visual indicator 870, and can also include and/or be coupled to a fluid and/or vacuum source 835. The visual indicator 870 can be coupled to (e.g., disposed between) the elongated tube 810 and the fluid and/or vacuum source 835.

The elongated tube 810 has a first end 811 (also referred to as a distal end) and a second end 813 (also referred to as a proximal end). Similarly as described above with respect to the elongated tube 210, the elongated tube 810 can optionally define any suitable number of lumens, such as a lumen 812. The lumen 812 can extend from the second end 813 to a location at or near the first end 811 of the elongated tube 810. The elongated tube 810 can define at least one opening 814 in fluid communication with the lumen 812 such that, when the first end 811 of the elongated tube 810 is disposed within a lumen or cavity of a patient (e.g., a stomach) and the second end 813 of the elongated tube 810 is disposed external to the patient, fluid can be delivered through the second end 813 of the elongated tube 810 and into the patient's lumen or cavity via the lumen 812 and the at least one opening 814. Similarly as described above with respect to the opening(s) 214, the at least one opening 814 can be defined in the first end 811 of the elongated tube 810 and/or in a sidewall of the elongated tube 810 near the first end 811. The at least one opening 814 (also referred to as fenestration(s)) can be any suitable size or shape (e.g., rounded, rectangular). Similarly as described with respect to the first end 211, the first end 811 optionally can be closed (e.g., a cap or cover can be coupled to the first end 811). In some embodiments, the elongated tube 810 can define one or more lumens in addition to the lumen 812, which can be, for example, a feeding lumen, that can each be used for fluid delivery and/or suction and/or for translation of any suitable components partially or fully through the elongated tube 810 (e.g., endoscopes or stiffening rods). In some embodiments, the elongated tube 810 can be solid except for the inflation lumen 815, which is described in more detail below, and thus may not include lumen 812 or additional lumens. In some embodiments, as shown in FIG. 20, the elongated tube 810 can optionally include one or more magnetic members 820 disposed near at or near the first end 811 of the elongated tube 810. The one or more magnetic members 820 can be the same or similar in structure and/or function as any of the magnetic members described herein, such as the magnetic member(s) 220.

The visual indicator 870 can be disposed external to the patient's body during placement of the distal end of the system within the patient's body (e.g., placement at or near a target location within the patient's body). For example, the visual indicator can be coupled to the second end 813 of the elongated tube 810 or disposed near or adjacent to the second end 813 of the elongated tube 810. The elongated tube 810 can be sufficiently long such that, when the first end 811 of the elongated tube 810 is disposed at a target location within a patient (e.g., within a target lumen or cavity), the second end 813 and the visual indicator 870 are disposed external to the patient and are within view of a user (e.g., an operator such as a clinician) of the system 800. The visual indicator 870 can be coupled to (e.g., disposed between) the elongated tube 810 and the fluid and/or vacuum source 835 such that the user who is handling (e.g., holding) the fluid and/or vacuum source 835 and facing a patient as the elongated tube 810 is partially disposed within the patient (e.g., through a nasal or oral orifice of the patient) can see the visual indicator 870 and/or a visual indication produced by the visual indicator 870. Thus, the visual indicator 870 can be coupled to the elongated tube 810 and the fluid and/or vacuum source 835 and disposed during use of the system 800 such that the visual indicator 870 and/or any visual indications generated by the visual indicator 870 are disposed in a line of sight between the user and the patient as the user performs a procedure to place the first end 811 of the elongated tube 810 within the patient) such that the user can easily a visual indication produced by the visual indicator 870 without needing to remove the user's hands from the system 800 (e.g., without needing to remove the user's hands from the fluid and/or vacuum source 835 as fluid is manually urged from and/or drawn into the fluid and/or vacuum source 835, such as a syringe, by the user as described below).

The system 800 can include an internal inflatable member 830 coupled to the elongated tube 810. The internal inflatable member 830 can be disposed at or sufficiently close to the first end 811 of the elongated tube 810 such that, when the first end 811 of the elongated tube 810 is properly positioned within the patient's body at a target location of the patient (e.g., within a stomach) and the second end 813 is disposed external to the patient (e.g., beyond the patient's nose or mouth through which the elongated tube 810 is disposed), the internal inflatable member 830 is also positioned at the target location. In some embodiments, the internal inflatable member 830 can be disposed distally to none, one, some, or all of the optional one or more openings 814 of the elongated tube 810 in communication with the lumen 812.

As described above, the elongated tube 810 can define an inflation lumen 815 in fluid communication with an interior of the internal inflatable member 830. The inflation lumen 815 can be transitioned between an unexpanded and expanded configuration (e.g., inflated and deflated) via fluid delivered and drawn through the inflation lumen 815. In some embodiments, the inflatable member 830 can surround a portion of the elongated tube 810 (e.g., can be coaxial with the elongated tube 810 in an expanded configuration). In some embodiments, the inflatable member 830 can extend from a portion of the elongated tube 810 asymmetrically relative to a central axis of the elongated tube 810. In some embodiments, the inflatable member 830 can be configured such that the inflatable member 830 has an outer diameter or maximum dimension perpendicular to a central axis of the elongated tube 810 in the expanded configuration such that the inflatable member 830 cannot fully expand to the expanded configuration in a non-target location within a patient's body (e.g., a patient's trachea, lungs, esophagus, and/or jejunum), but can fully expand to the expanded configuration in a target location within a patient's body (e.g., a patient's stomach).

The internal inflatable member 830 can be fluidically coupled with the visual indicator 870 via the inflation lumen 815 of the elongated tube 810. The visual indicator 870 can be configured to provide a visible indication (e.g., visible to the user while the user is positioned at or near the second end 813 of the elongated tube 810 and/or is holding and/or manipulating the system 800 relative to a patient) in response to a pressure within the internal inflatable member 830 and the inflation lumen 815 increasing above a threshold pressure. As described above, for example, the internal inflatable member 830 can be configured (e.g., shaped and sized) such that the internal inflatable member 830 can transition between the unexpanded configuration and the expanded configuration, and the internal inflatable member 830 can have a shape and size (e.g., outermost dimension(s), such as diameter) in the expanded configuration that is equal to or smaller than an internal space defined by a cavity or lumen associated with a target location of the patient's body (e.g., a stomach) and that is greater than an internal space defined by a cavity or lumen associated with a non-target location of the patient's body (e.g., an esophagus or trachea). Thus, when the internal inflatable member 830 is in a location within the patient in which the internal inflatable member 830 is restricted from fully expanding to the expanded configuration (due to the geometry of the non-target location relative to the internal inflatable member 830), the continued introduction of fluid to the internal inflatable member 830 and/or to an interior space of the system 800 collectively defined at least in part by the internal inflatable member 830 and the inflation lumen 815 can cause the pressure of the fluid within the internal inflatable member 830 and/or within the interior space of the system 800 to increase above a threshold pressure. When fluidically coupled to the space defined by the internal inflatable member 830 and the inflation lumen 815, the visual indicator 870 can indicate a pressure of fluid within the internal inflatable member 830 and/or the interior space of the system 800 and/or that the pressure of fluid within the internal inflatable member 830 and/or the interior space of the system 800 has risen above the threshold pressure.

In some embodiments, the visual indicator 870 can be configured to transition between providing a first visual indication associated with a pressure within the system being below a threshold pressure, and a second visual indication associated with a pressure within the system increase above the threshold pressure. In some embodiments, the visual indicator 870 can provide the first visual indication by being disposed in a first location and/or configured in a first shape or size, and the visual indicator 870 can provide the second visual indication by being disposed in a second location and/or configured in a second shape or size. In some embodiments, the first visual indication can be a lack of indication and/or a lack of change in configuration (e.g., shape and/or size).

The visual indicator 870 indicating that the pressure of fluid within the internal inflatable member 830 and/or the interior space of the system 800 is above a threshold pressure can signal to the user that the internal inflatable member 830 is in a location that is preventing the internal inflatable member 830 from fully expanding, causing the volume of the internal inflatable member 830 to be restricted to a smaller volume than a volume of the internal inflatable member 830 in the fully expanded configuration. Thus, the visual indicator 870 indicating that the pressure of fluid within the space is above a threshold pressure can signal to the user that the internal inflatable member 830 is disposed in a non-target location, and therefore needs to be moved to be disposed in a target location within the patient's body in which the internal inflatable member can fully expand. In some embodiments, if the internal inflatable member 830 is within the patient and a volume of fluid sufficient to expand the internal inflatable member 830 to the fully expanded configuration has been delivered into the internal inflatable member 830 and/or the interior space of the system 800, an alternative indication or lack of indication of the visual indicator 870 can signal to the user that the internal inflatable member 830 and/or the distal end of the elongated tube 810 is disposed in the target location. For example, the visual indicator 870 can indicate that the pressure has not increased above the threshold pressure and/or that the internal inflatable member 830 was able to fully expand within the patient by not providing an indication or by not changing with respect to an indication provided, such as by not changing in configuration or shape.

In some embodiments, the fluid and/or vacuum source 835 can be fluidically coupled to the inflation lumen 815. In some embodiments, the fluid and/or vacuum source 835 can define a reservoir of gaseous fluid and can be manipulated (e.g., squeezed or pressed by a healthcare provider) to urge the gaseous fluid into the inflation lumen 815 and to the internal inflatable member 830 to transition the internal inflatable member 830 from the unexpanded to the expanded configuration. In some embodiments, the fluid and/or vacuum source 835, the inflation lumen 815, the visual indicator 870, and the internal inflatable member 830 can form a closed fluid system such that gaseous fluid can be delivered from the fluid and/or vacuum source 835 into the inflation lumen 815, and into the internal inflatable member 830 to fully expand the internal inflatable member 830, and then, the fluid and/or vacuum source 835 can expand to draw the fluid from the internal inflatable member 830, through the inflation lumen 815, and into the fluid and/or vacuum source 835.

In some embodiments, due to the fluid and/or vacuum source 835, the inflation lumen 815, the visual indicator 870, and the internal inflatable member 830 forming a closed fluid system, if the internal inflatable member 830 is disposed in a location (e.g., a trachea, esophagus, lung, or small bowel) that restrains the internal inflatable member 830 and does not allow the internal inflatable member 830 to fully expand to the expanded configuration, the visual indicator 870 will provide a visual indication to the user that the internal inflatable member 830, and thus the first end 811 of the elongated tube 810, is restrained and in the wrong location within the patient. In response to the visual indication provided by the visual indicator 870, the user can cease delivery of fluid from (e.g., cease applying pressure to) the fluid and/or vacuum source 835 and manipulate (e.g., translate) the elongated tube 810 to move the internal inflatable member 830 and the first end 811 to a new location within the patient to test.

In some embodiments, the fluid and/or vacuum source 835 can include a syringe defining a reservoir and couplable to the inflation lumen 815 such that the syringe can be manipulated to push fluid (e.g., a bolus) to the internal inflatable member 830 via the inflation lumen 815 and to draw fluid from the internal inflatable member 830 via the inflation lumen 815. In some embodiments, the fluid and/or vacuum source 835 can include a flexible bulb defining a reservoir sealable to the elongated tube 810 (e.g. via any suitable fluid connector and/or adhesive). The flexible bulb can be squeezed to push gaseous fluid (e.g., a bolus of gaseous fluid) into the inflation lumen 815 and can be biased toward an expanded configuration such that, when the flexible bulb is released, gaseous fluid (e.g., the gaseous fluid previously pushed out of the flexible bulb) is drawn into the flexible bulb.

In some embodiments, the visual indicator 870 can be configured to provide a plurality of indications. In some embodiments, the plurality of indications can include a first indication, a second indication, and one or more intermediate indications configured to be indicated by the visual indicator 870 between indication of the first indication and the second indication as fluid is delivered to the internal inflatable member 830 and/or the interior space of the system 800 based on the shape and/or size of an internal space defined by a location of the patient's body within which the internal inflatable member 830 is disposed as the fluid is delivered. In some embodiments, each indication of the plurality of indications associated with the interior pressure within the system increasing above a different pressure or being within a different pressure sub-range within a range of pressures. In some embodiments, the visual indicator 870 can include at least a portion configured to transition between a first configuration and a second configuration (e.g., a first length and a second length and/or between a first shape and a second shape). Each of the first configuration, the second configuration, and any suitable number of intermediate configurations therebetween can be associated with a different internal pressure threshold or sub-range of the interior space of the system 800 (e.g., within the inflation lumen 815 and/or the internal inflatable member 830). For example, as described in more detail below, in some embodiments, the visual indicator 870 can include a coiled inflatable member that is in fluid communication with the inflation lumen 815 and the internal inflatable member 830, and the coiled inflatable member can have a first coiled configuration, a second fully uncoiled (e.g., straight and/or tubular) configuration, and one or more partially coiled configurations serially assumed by the coiled inflatable member as the coiled inflatable member transitions between the coiled configuration and the fully uncoiled configuration (e.g., a 25% uncoiled configuration, a 50% uncoiled configuration, and a 75% uncoiled configuration). Each of the partially coiled configurations can be associated with a different indication provided by the visual indicator 870 (e.g., a different internal pressure threshold reached within the system 800 and/or a different internal volume of the system 800 needed to maintain an internal pressure of the internal inflatable member 830 and/or the system 800 within a range, such as an internal pressure at which the internal inflatable member 830 does not apply an unsafe force against an internal tissue wall of the patient).

In some embodiments, one or more of the intermediate configurations assumable by the visual indicator 870 between the first and second configurations (e.g., the partially coiled configurations) can be associated with the distal end of the elongated tube 810 and/or the internal inflatable member 830 being disposed at the target location, and the second configuration (e.g., the fully uncoiled configuration) can be associated with the distal end of the elongated tube 810 and/or the internal inflatable member 830 being disposed at a non-target location. In some embodiments, the first configuration can be associated with the distal end of the elongated tube 810 and/or the internal inflatable member 830 being disposed at the target location, and one or more of the configurations assumable by the visual indicator 870 between the first and second configurations (e.g., the partially coiled configurations) and/or the second configuration (e.g., the fully uncoiled configuration) can be associated with the distal end of the elongated tube 810 and/or the internal inflatable member 830 being disposed at a non-target location. In some embodiments, the visual indicator 870 forming the shape of one or more of the configurations between the first and second configurations (e.g., the partially coiled configurations) can indicate to the user that the elongated tube 810 and/or the internal inflatable member 830 is disposed at a non-target location within which the internal inflatable member 830 should not be further inflated to avoid risk of damaging internal structure (e.g., tissue or organs) of the patient at the non-target location.

In some embodiments, the visual indicator 870 can be electronic. In some embodiments, the visual indicator 870 can include a pressure sensor in fluid communication with the inflation lumen 815 and configured to sense a pressure of fluid in contact with the visual indicator 870 and generate one or more indications based on the sensed pressure. The pressure sensor can be electronic and digital or analog. In some embodiments, the one or more indications can be provided, for example, via one or more light-emitting diodes (LEDs), with each associated with a different pressure threshold or subrange of fluid within the system 800. In some embodiments, the visual indicator 870 can be configured to produce one or more audible, tactile, odoriferous, and/or tastable indication(s) as an alternative to or in addition to producing one or more visual indications. In some embodiments, each of these sensory indications are distinct from those typically experienced by a clinician while performing a procedure on a patient's body (e.g., a procedure including placement of a distal end of an elongated tube within a patient's body) such that the indication is easily recognizable by the user. Thus, the visual indicator 870 described herein can alternatively be referred to as a “restraint indicator,” an “internal restriction indicator,” an “internal cross-section indicator,” an “expansion restriction indicator,” a “target location indicator,” a “non-target location indicator,” an “internal space size indicator,” and/or an “indicator,” and can be configured to produce non-visual indications in addition to as an alternative to producing visual indications in the same or similar ways as described herein with respect to producing visual indications. In some embodiments, the indicator 870 described herein is not configured to produce visual indications, but is configured to produce one or more other sensory indications in place of the visual indications described herein. In some embodiments, the indicator 870 can be configured to provide a combination of any of the sensory indications perceptible by a user (e.g., clinician) (e.g., a combination of two, three, four or more types of indications), which can be provided serially and/or simultaneously and can be associated with one or more configurations of the internal inflatable member 830 and/or one or more pressure thresholds associated with fluid in an interior space of the internal inflatable member 830 and/or the system 800. For example, the indications provided can be a combination of a visual indication and a tactile indication, a visual indication and an odoriferous indication, a tactile indication and testable indication, etc.

In some embodiments, the visual indicator can optionally include an external inflatable member 876 that is configured to transition from an unexpanded to an expanded configuration in response to the pressure within the system 800 (e.g., within the inflation lumen 815 and the internal inflatable member 830) rising above a threshold pressure (e.g., due to the internal inflatable member 830 being restrained from fully expanding within a patient). The external inflatable member 876 can be configured to have a first shape and/or size in the first configuration and a second shape and/or size in the second configuration that is visually distinguishable from the first shape and/or size. For example, the external inflatable member 876 can have a first length, first width, and/or first height in the first configuration, and can have a second length, second width, and/or second height in the second configuration that is greater than the first length, first width, and/or first height, respectively. In some embodiments, the external inflatable member 876 can have a collapsed configuration having a first length in the first configuration, and can have an elongated configuration having a second length that is longer than the first length (e.g., one, two, three, four, or more times as long) in the second configuration. In some embodiments, the external inflatable member 876 can be in a coiled or rolled configuration in the first configuration and in an uncoiled or unrolled configuration in the second configuration.

In some embodiments, the visual indicator 870 can include a collapsing member coupled to the external inflatable member 876 (e.g., disposed within the external inflatable member 876 or coupled to an external surface of the external inflatable member 876) and biased toward a collapsed configuration to assist in keeping the external inflatable member 876 tidy and in a non-obstructing configuration when in the unexpanded configuration. For example, in some embodiments, the visual indicator 870 can include a coil coupled to the external inflatable member 876. The coil can be configured to be transitioned between a coiled configuration and an expanded configuration by the external inflatable member 876 as the external inflatable member 876 transitions between the unexpanded and the expanded configuration. The coil can be biased toward the coiled configuration such that the external inflatable member 876 stays in the unexpanded configuration, which can be coiled, prior to inflation and/or such that the external inflatable member 876 returns to the first configuration after deflation.

In some embodiments, the visual indicator 870 can include a housing having an inlet, an outlet, a port, and an interior space. The external inflatable member 876 (e.g., a balloon) can be coupled to the port and fluidically coupled to the inlet and the outlet via the interior space. The external inflatable member 876 can transition between the unexpanded configuration (also referred to as a first, uninflated configuration) and the expanded configuration (also referred to as a second, inflated configuration) in response to an increase in pressure within the interior space. The inlet of the visual indicator 870 can be configured to be fluidically and mechanically coupled to the fluid and/or vacuum source 835 (e.g., a syringe). The outlet can be mechanically coupled to the elongated tube 810 and fluidically coupled to the inflation lumen 815 such that the interior of the visual indicator 870 is fluidically coupled to the internal inflatable member 830 disposed near the distal end of the elongated tube 810.

In some embodiments, the visual indicator 870 can include a valve disposed within the port and/or adjacent an opening of the external inflatable member 876. The valve can be configured to allow fluid to flow into the external inflatable member 876 only when the pressure within the system is above a threshold pressure. Thus, when a volume of fluid (e.g., a bolus) that would be sufficient to transition the internal inflatable member 830 from the unexpanded to the expanded configuration is delivered from the fluid and/or vacuum source 835 into the inflation lumen 815, the external inflatable member 876 will only expand if the internal inflatable member 830 is restricted from fully expanding (e.g., by the patient's body), causing a portion of the fluid to flow through the valve and into the external inflatable member 876 to cause the external inflatable member 876 to at least partially expand.

In some embodiments, the system 800 can be assembled by the user prior to use with a patient. The visual indicator 870 can be fluidically coupled to the inflation lumen 815 and the internal inflatable member 830 of the elongated tube 810 and to the fluid and/or vacuum source 835. As described above, in some embodiments, the visual indicator 870 can include a housing including an outlet couplable to the inflation lumen 815 (e.g., insertable into the inflation lumen 815 or couplable via any suitable fluid connector or fitting, such as a Luer connection) and an inlet configured to be coupled to the fluid and/or vacuum source 835 (e.g., via any suitable fluid connector or fitting, such as a Luer connection).

In some embodiments, after the initial assembly of the system 800, which can form a closed fluid system when assembled, the fluid and/or vacuum source 835 (e.g., a syringe or bulb) can be manipulated to draw or squeeze all or substantially all gaseous fluid (e.g., air) out of the remainder of the system (e.g., the internal inflatable member 830, the inflation lumen 815, and/or the visual indicator 870). The fluid and/or vacuum source 835 can then be disconnected from the visual indicator 870. While decoupled from the visual indicator 870, the fluid and/or vacuum source 835 can be manipulated such that a first volume of gaseous fluid (e.g., air) is drawn into the fluid and/or vacuum source 835. The syringe can then be reconnected to the inlet of the visual indicator 870 while the first volume of gaseous fluid is disposed within the fluid and/or vacuum source 835. The fluid and/or vacuum source 835 can optionally be further manipulated to draw a second volume of gaseous fluid (e.g., air) into the fluid and/or vacuum source 835 from the internal inflatable member 830, the inflation lumen 815, and/or the visual indicator 870 (e.g., to remove any fluid that entered the internal inflatable member 830, the inflation lumen 815, and/or the visual indicator 870 while the syringe was disconnected from the visual indicator 870). The second volume of gaseous fluid can combine with the first volume of gaseous fluid within the fluid and/or vacuum source 835.

In some embodiments, the first volume of gaseous fluid is about 45 ml and the second volume of gaseous fluid is about 5 ml such that the total amount of fluid within the fluid and/or vacuum source 835 at this step (and within the closed system 800 at this step and during subsequent procedure steps) is about 50 ml. In some embodiments, the total amount of fluid within the closed system 800 is a volume sufficiently low for the fluid and/or vacuum source 835 to fully deliver the first volume of gaseous fluid and the second volume of gaseous fluid so that the internal inflatable member 830 is fully expanded within a target cavity or lumen without the internal pressure of the system 800 rising above a pressure threshold that causes the visual indicator 870 to provide an indication associated with the internal inflatable member 830 being in the non-target location (e.g., without the external inflatable member 876 expanding), but the volume is sufficiently high for the visual indicator 870 to provide the indication (e.g., for the external inflatable member 876 to expand) in response to gaseous fluid being fully delivered from the fluid and/or vacuum source 835 into the remainder of the system 800 when the internal inflatable member 830 is in a non-target location within the patient that includes and/or is formed of a structure (e.g., shape and/or size) that prevents the internal inflatable member 830 from fully expanding.

In some embodiments, if the internal inflatable member 830 is disposed in a location (e.g., a target location) defining a space sufficiently large for the internal inflatable member 830 to expand to the fully expanded configuration (e.g., a size equal to or smaller than the space defined by the location), the entire deliverable fluid volume of the fluid and/or vacuum source 835 (e.g., from a syringe or a bulb) can be delivered from the fluid and/or vacuum source 835 into the interior space defined by a remainder of the system 800 (e.g., an interior space at least partially defined by the internal inflatable member 830 and/or the elongated tube 810) without the visual indicator 870 (e.g., the external inflatable member 876) transitioning from the first configuration (e.g., changing and/or expanding). For example, a valve of the visual indicator 870 can remain closed due to the fluid pressure within the system 800 in fluid communication with the valve remaining below a fluid pressure threshold associated with the valve opening, and thus no fluid travels beyond the valve of the visual indicator 870 (e.g., into the external inflatable member 876).

In some embodiments, if the internal inflatable member 830 is disposed in a location (e.g., a non-target location) defining a space that is sufficiently small to restrain the internal inflatable member 830 from fully transitioning to the expanded configuration as fluid is delivered from the fluid and/or vacuum source 835 into the interior space defined by a remainder of the system 800, in response to the internal inflatable member 830 contacting the structure (e.g., tissue) defining the space of the location and being restrained from further expanding, the pressure within the remainder of the system 800 will rise as additional fluid is delivered to the remainder of the system 800 since the volume of the remainder of the system 800 is restrained from increasing by the location within which the internal inflatable member 830 is disposed. As the pressure increases, the visual indicator 870 can be configured to provide one or more indications that the pressure is increasing and/or has increased beyond a threshold pressure, and thus that the internal inflatable member 830 is restrained from expanding or further expanding at the current location. In some embodiments, as a result of the pressure increase, a valve of the visual indicator 870 can open, and the visual indicator 870 can begin to transition from the first configuration toward a second configuration (e.g., the external inflatable member 876 can begin to expand) when only a portion of the fluid volume deliverable from the fluid and/or vacuum source 835 has been delivered to the remainder of the system 800. As additional fluid is delivered from the fluid and/or vacuum source 835, the fluid can continue to flow (e.g., overflow) to the visual indicator 870 (e.g., to inflate the external inflatable member 876). In some embodiments, the external inflatable member 876 of the visual indicator 870 can be configured to continue to expand to increase the volume of the remainder of the system 800 as additional fluid is delivered from the fluid and/or vacuum source 835 such that a fluid pressure within the remainder of the system 800 is maintained below a pressure threshold (e.g., below a pressure threshold in which the fluid pressure may cause the internal inflatable member 830 to apply a damaging force against the tissue of the location within which the internal inflatable member 830 is disposed).

In some embodiments, the volume of fluid delivered (or sub-range of volume) from the fluid and/or vacuum source 835 when the visual indicator 870 provides the initial indication of a change in pressure (e.g., when the external inflatable member 876 begins expanding) can indicate and/or be associated with a particular size and/or shape (e.g., a cross-sectional area or dimension and/or range of areas or dimensions) of the structure of the location constraining the internal inflatable member 830 from further expanding. Thus, the volume delivered and/or still remaining in the fluid and/or vacuum source 835 at the time during delivery when the visual indicator 870 provides the initial indication of a change (e.g., the external inflatable member 876 begins inflating) can be associated with a particular non-target region of the body. For example, a first volume delivered at the time of the initial indication of a change can be associated with a first location (e.g., a first non-target location), and thus can indicate that the internal inflatable member 830 (and thus the first end 811 of the elongated tube 810) is disposed at a first location within the body of the patient. A second volume delivered at the time of the initial indication of a change can be associated with a second location (e.g., a second non-target location), and thus can indicate that the internal inflatable member 830 (and thus the first end 811 of the elongated tube 810) is disposed at a second location within the body of the patient. The second non-target location can include a structure defining a larger space for expansion of the internal balloon than the first non-target location (e.g., can have a larger internal cross-sectional area and/or dimensions such as diameter), such that a greater volume of fluid can be delivered from the fluid and/or vacuum source 835 before the visual indicator 870 provides the initial indication of a change when the internal inflatable member 830 is disposed in the second location than the first location. In some embodiments, for example, the second location can be a trachea of a patient and the first location can be an esophagus. In some embodiments, the initial indication of the visual indicator 870 that pressure is increasing (e.g., the initial partial expansion of the external inflatable member 876) can signal to the user that the internal inflatable member 830 is in a location of a particular cross-sectional area and/or maximum dimension (e.g., diameter), and the user can discontinue the delivery of fluid to the remainder of the system 800.

In some embodiments, rather than using the systems and methods described herein to seek a target location being associated with a fully expanded configuration of the internal inflatable member 830, a system, such as any of the systems described herein, can be used to identify a particular region of a subject's body within which an inflatable member and/or distal end of an elongated tube is disposed (e.g., compared to one or more other regions). Thus, the target location can be a location that does not allow for full expansion of the internal inflatable member 830 and a non-target location can be a location that does allow for fully expansion of the internal inflatable member 830. For example, in some embodiments, rather than target location being associated with fully expanded configuration of the internal inflatable member 830, the target location can be associated with a particular partially inflated volume or volume range of the internal inflatable member 830. For example, the target location can define an internal space that is larger than a first non-target location's internal space and smaller than a second non-target location's internal space, so if the full source volume is delivered without the visual indicator 870 providing an indication of a change in pressure (e.g., without external inflatable member 876 inflating), the user can understand that the internal inflatable member 830 is in the second non-target location (e.g., a stomach), and if a first volume of fluid is delivered at the time the visual indicator 870 provides an indication of a change in pressure, the user can understand that the internal inflatable member 830 is in the first non-target location (e.g., the esophagus). If a second volume of fluid is delivered at time the visual indicator 870 provides an indication of a change in pressure, the user can understand that the internal inflatable member 830 is in the target location (e.g., trachea).

In some embodiments, a single indicator of the visual indicator 870, such as an inflatable member such as the external inflatable member 876, can provide a plurality of indications, with each indication associated with different amounts of restriction on the internal inflatable member 830 associated with different locations within the patient. Thus, based on delivery of a particular volume of from the fluid and/or vacuum source 835, the indication provided by the external inflatable member 876 can indicate to the user the size of the location and/or location of the inflatable member 830. For example, if a particular partial volume of fluid is delivered from the fluid and/or vacuum source 835 when the inflatable member 830 is at a first location and the external inflatable member 876 transitions to a first configuration (e.g., a first size having a first volume), and the same partial volume of fluid is delivered from the fluid and/or vacuum source 835 when the inflatable member 830 is at a second location and the inflatable member 830 transitions to a second configuration (e.g., a second size having a second volume), these intermediate indications can be associated with the size of each of the first and second location, relative sizes of the first and second location, and/or identify of the first and second location. Additionally, in some embodiments, the intermediate configuration of the external inflatable member 876 after a partial delivery of fluid volume can indicate that fluid delivery should be discontinued because the internal inflatable member 830 is constrained from further expanding.

In some embodiments, the visual indicator 870 can include more than one external inflatable member 876. In some embodiments, each of the external inflatable members 876 can be configured to change in configuration (e.g., inflate) in response to different fluid pressures within the system 800. For example, a first inflatable member 876 can expand at a lower threshold fluid pressure than a second inflatable member 876. Thus, in some embodiments, the second inflatable member 876 can function as a receiver of fluid overflow when the first inflatable member 876 is partially or fully inflated (e.g., maintaining a fluid pressure within the system 800 below a threshold that may be damaging to the system 800 or the patient). In some embodiments, the configuration of each of the first external inflatable member 876 and the second external inflatable member 876 associated with different volumes of fluid delivery can indicate the size of the location within the body within which the internal inflatable member 830 is disposed.

In some embodiments, the first inflatable member 876 may not be associated with a valve and may be in constant fluid communication with the internal inflatable member 830 via the elongated tube 810, and the second inflatable member 876 can include a valve configured to open in response to a fluid pressure within the system increasing beyond a threshold pressure. Thus, the first inflatable member 876 can change in configuration (e.g., expand due to inflation) similarly to the internal inflatable member 830 and be representative of the size of the internal inflatable member 830 within the patient. Once the internal inflatable member 830 is restrained from further expansion within the patient due to the structure of the patient at the location of the internal inflatable member 830, additional delivery of fluid to the remainder of the system 800 can cause the fluid pressure to increase and the valve to open such that additional fluid delivered at least partially flows to the second inflatable member 876, indicating the restriction on the internal inflatable member 830 to the user. In some embodiments, the first inflatable member and the second inflatable member can have different elasticities such that, as the additional fluid is delivered to the remainder of the system 800, more fluid delivered to the second inflatable member than the first inflatable member.

In some embodiments, two or more external inflatable members 876 can be configured to inflate in series, with the inflation of each additional external inflatable members 876, in combination with an observation of the volume of fluid delivered from the source 835 (e.g., partial or full delivery), providing an indication of the size of the location within which the internal inflatable member 830 is disposed. The fewer external inflatable members 876 that are inflated for a particular volume of fluid delivered, the larger the space within which the internal inflatable member 830 is disposed compared to a location at which more external inflatable members 876 are inflated in response to the same volume of fluid being delivered.

In some embodiments, each of the internal inflatable member 830 and/or the one or more external inflatable members 876 included in the visual indicator 870 can be elastic, inelastic, or a combination thereof, such that the shape of each inflatable member can be controlled as it expands and each inflatable member can optionally be configured to have a maximum volume based on the fluid pressures possibly attained within the system 800. In some embodiments, at least one of the external inflatable member 876 can have the same expansion properties and/or characteristics as the internal inflatable member 830.

As described above, in some embodiments, rather than including more than one external inflatable member 876, the system 800 can be configured to produce any suitable combination of sensory indications that can be used to provide information about the system 800 at different times and/or internal pressures during operation of the system 800. For example, rather than having a first external inflatable member 876 that inflates in response to a first internal fluid pressure of the system 800 and a second external inflatable member 876 that inflates in response a second internal fluid pressure of the system 800, the system can include an auditory indication associated with the first internal fluid pressure and a visual indication (e.g., external inflatable member 876 expansion) at a second internal fluid pressure, or an odiferous indication associated with the first internal fluid pressure and an auditory indication associated with the second internal fluid pressure. In some embodiments, two or more sensory indications can be produced by the system 800 simultaneously, such as both a visual and tactile indication being produced at a second internal fluid pressure, with only the tactile indication being produced at a first internal fluid pressure.

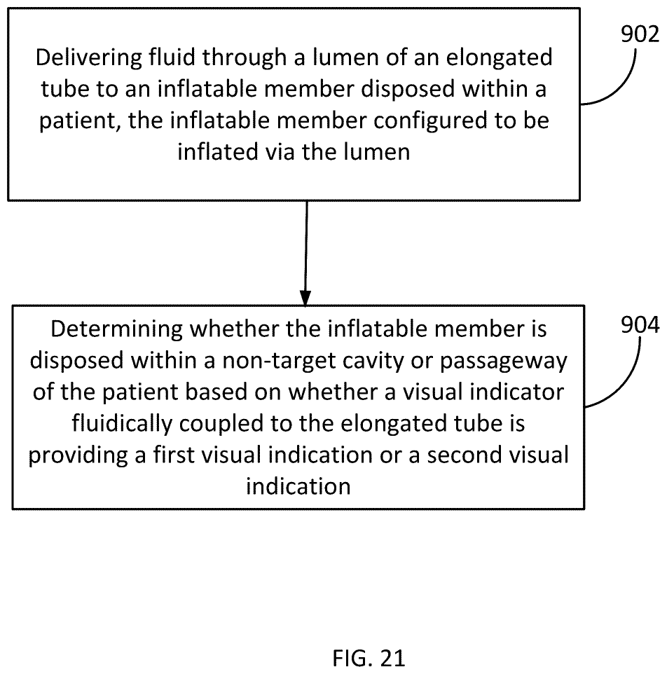

FIG. 21 is a flow chart illustrating a method 900 of using any of the systems and/or assemblies described herein, such as the system 800. The method 900 includes delivering, at 902, fluid through a lumen (e.g., the inflation lumen 815) of an elongated tube (e.g., elongated tube 810) to an internal inflatable member (e.g., internal inflatable member 830) disposed within a patient. The internal inflatable member is configured to be inflated via the lumen. The method 900 further includes determining, at 904, whether the internal inflatable member is disposed at a non-target location (e.g., within a non-target cavity or passageway such as a trachea, esophagus or any constricted area where end of tube is not supposed to be) of the patient based on whether an external visual indicator (e.g., the visual indicator 870) is providing a first visual indication or a second visual indication. For example, the visual indicator can include an external inflatable member fluidically coupled to the elongated tube that has a first configuration associated with the first visual indication and a second configuration associated with the second visual indication.

The delivering can optionally include applying pressure to a source of the fluid (e.g., the fluid and/or vacuum source 835, such as a syringe or bulb), the source being in fluid communication with the internal inflatable member via the lumen. The delivering can optionally include delivering the entire or substantially the entire volume of the source of fluid into the remainder of the system. The determining can optionally include visually observing whether the external inflatable member has transitioned to or towards a second configuration (e.g., has expanded or inflated) in response to the delivered fluid. For example, in some embodiments, an expansion of the external inflatable member from the first configuration to or toward the second configuration can be observed and the amount of expansion can be based on a volume of the internal inflatable member when the internal inflatable member becomes resistant to further increasing. The expansion of the external inflatable member signifies to the user that the internal inflatable member is constrained by the patient's body and is unable to be further expanded and/or faces increased resistance to being further expanded compared to the internal inflatable member's initial partial expansion.

In some embodiments, upon determining that the inflatable member is disposed in a non-target location (e.g., due to observing an indication provided by the visual indicator such as the expansion of an external inflatable member), the elongated tube can be manipulated to relocate the internal inflatable member. For example, gaseous fluid can be partially or substantially completely drawn from the visual indicator (e.g., from the external inflatable member through the valve thereof), the inflation lumen, and/or the internal inflatable member into the fluid and/or vacuum source, causing the internal inflatable member to collapse to an unexpanded configuration if it partially expanded, and the elongated tube can be translated relative to the patient such that the distal end and the internal inflatable member are in a new location to test. The delivering can be performed again to deliver gaseous fluid from the fluid and/or vacuum source such that it flows into the inflation lumen and to the internal inflatable member. If the gaseous fluid is fully delivered without the visual indicator providing an indication that the internal inflatable member is constrained (e.g., without the external inflatable member expanding), the user can determine that the internal inflatable member, and thus the distal end of the elongated tube, are in the target location (e.g., a target cavity or passageway such as the stomach of the patient). If the gaseous fluid is partially or fully delivered and the visual indicator provides an indication that the internal inflatable member is constrained (e.g., the external inflatable member expands due to diversion of air from the internal inflatable member through a valve and into the external inflatable member), the user can determine that the internal inflatable member, and thus the distal end of the elongated tube, are in a non-target location. If determined to be in a non-target location, the user can repeat the above steps to relocate the internal inflatable member and distal end of the elongated tube to a new location to test.

In some embodiments, upon determining that the internal inflatable member is not disposed in a non-target location, and thus determining that the internal inflatable member is disposed in the target location, liquid fluid can be provided through a feeding lumen of the elongated tube to the target location.

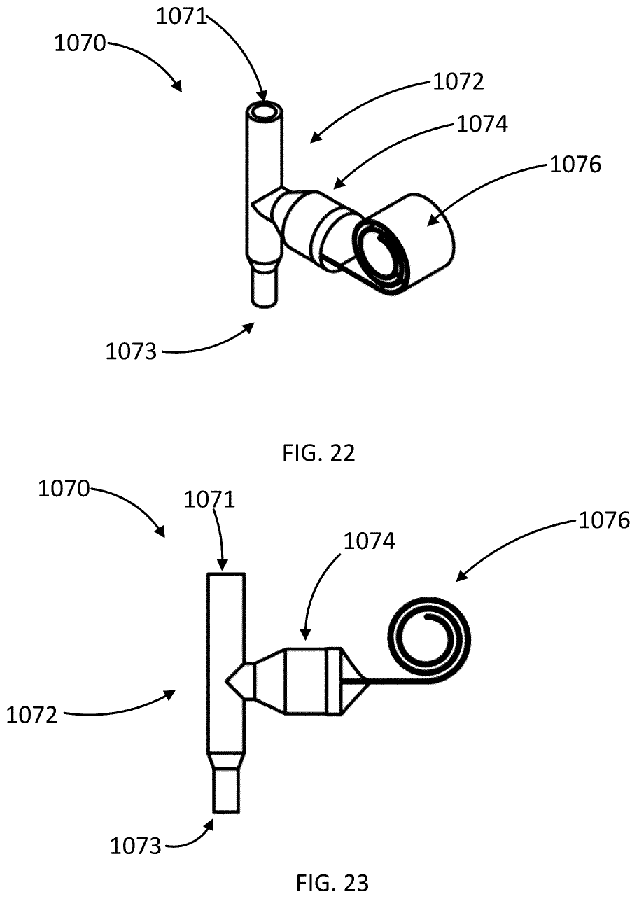

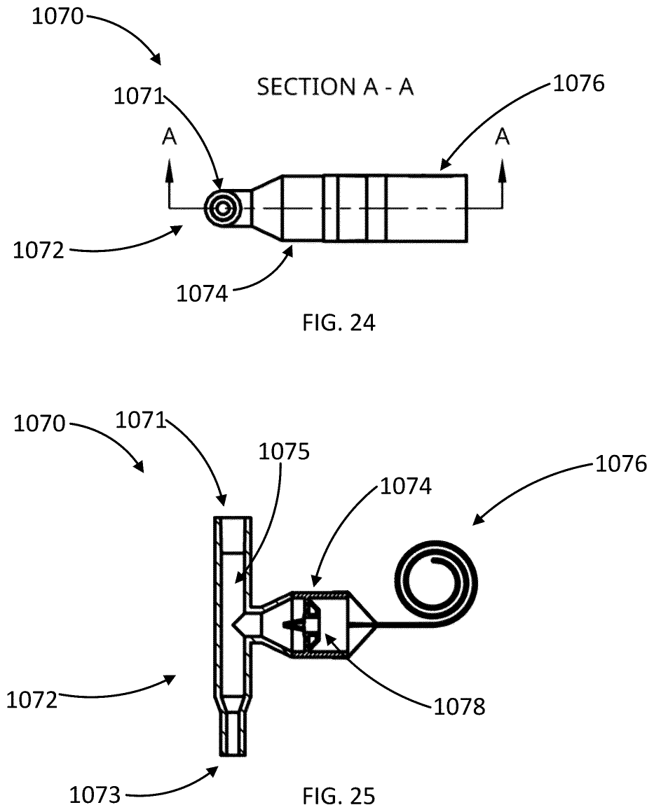

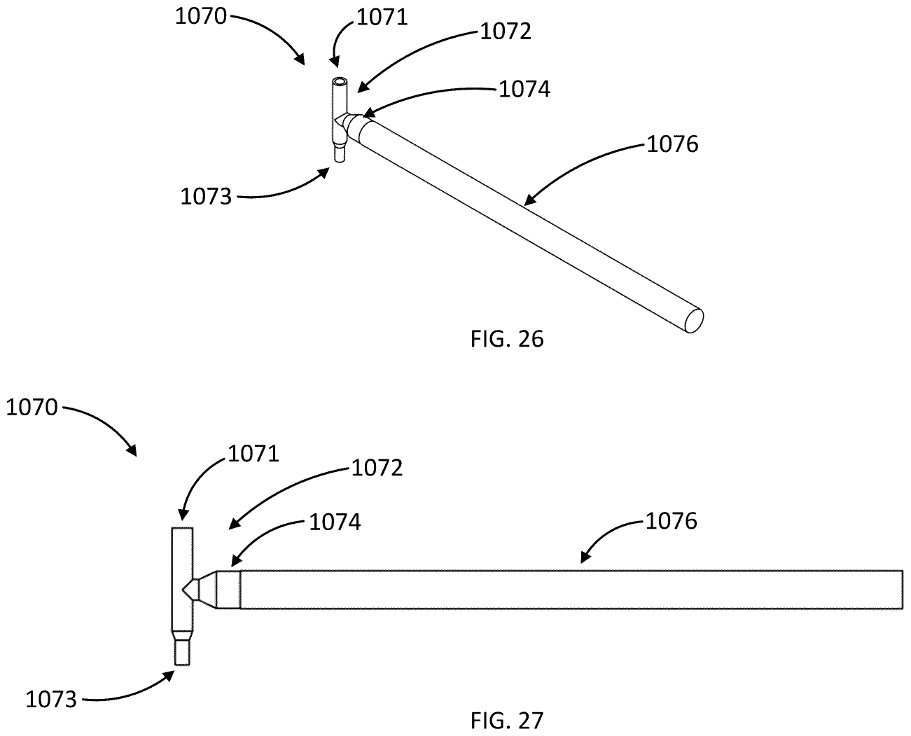

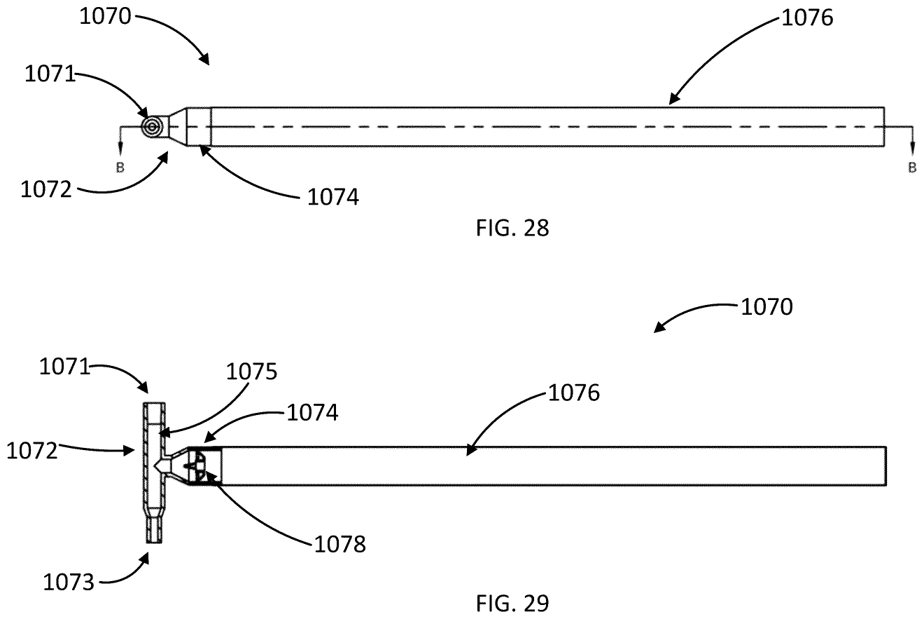

FIGS. 22-29 are schematic illustrations of a visual indicator 1070. Specifically, FIG. 22 is a perspective view, FIG. 23 is a side view, and FIG. 24 is a top view of the visual indicator 1070 in a first, unexpanded configuration. FIG. 25 is a cross-sectional view taken along line A-A in FIG. 24. FIG. 26 is a perspective view, FIG. 27 is a side view, and FIG. 28 is a top view of the visual indicator 1070 in a second, expanded configuration. FIG. 29 is a cross-sectional view taken along line B-B in FIG. 28.

The visual indicator 1070 can be the same or similar in structure and/or function to any of the visual indicators described herein, such as the visual indicator 870. For example, the visual indicator 1070 includes a housing 1072 and an inflatable member 1076. The housing 1072 includes an inlet 1071, an outlet 1073, and a port 1074, and defines an interior space 1075. The inlet 1071 can be configured to be coupled to a vacuum and/or fluid source (e.g., a syringe) via any suitable coupling or fitting, such as a Luer connection. The outlet 1073 can be configured to be coupled to a proximal end of an elongated tube such that the interior space 1075 is in fluid communication with a lumen of the elongated tube. For example, the outlet 1073 can be disposed within a proximal end of the lumen of the elongated tube.

The inflatable member 1076 (e.g., a balloon) is coupled to the port 1074 and fluidically coupled to the inlet 1071 and the outlet 1073 via the interior space 1075. The inflatable member 1076 can transition between the unexpanded configuration (also referred to as a first, uninflated configuration) and the expanded configuration (also referred to as a second, inflated configuration) in response to an increase in pressure within the interior space 1075. Although shown as being coiled in the unexpanded configuration and tubular in the expanded configuration, the inflatable member 1076 can have any suitable shape in the unexpanded configuration and the expanded configuration.

As shown in FIGS. 25 and 29, the visual indicator 1070 includes a valve 1078 disposed within the port 1074 and configured to selectively allow fluid to flow between the interior space 1075 and the inflatable member 1076 (e.g., when a pressure of fluid within the interior space 1075 is above a threshold pressure).

Although not shown, the inflatable member 1076 can include a coil configured to transition between a coiled configuration associated with the shape of the inflatable member 1076 in FIGS. 22-25 and an elongated, substantially straight configuration associated with the shape of the inflatable member 1076 in FIGS. 26-29 depending on whether the inflatable member 1076 is unexpanded or expanded. The coil can be biased toward the coiled configuration such that, when the inflatable member 1076 is in the unexpanded configuration, the inflatable member 1076 forms a coiled shape under the control of the coil as shown in FIGS. 22-25.

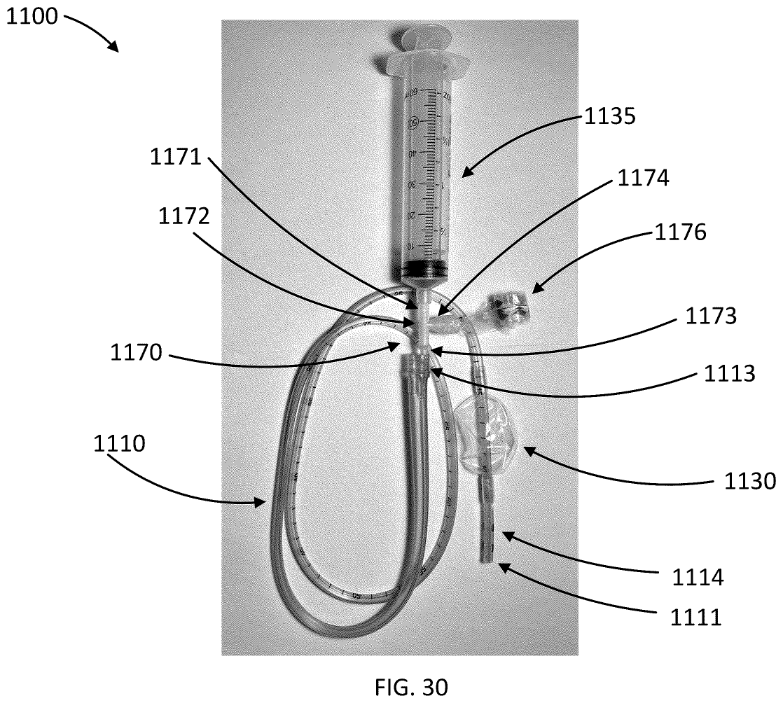

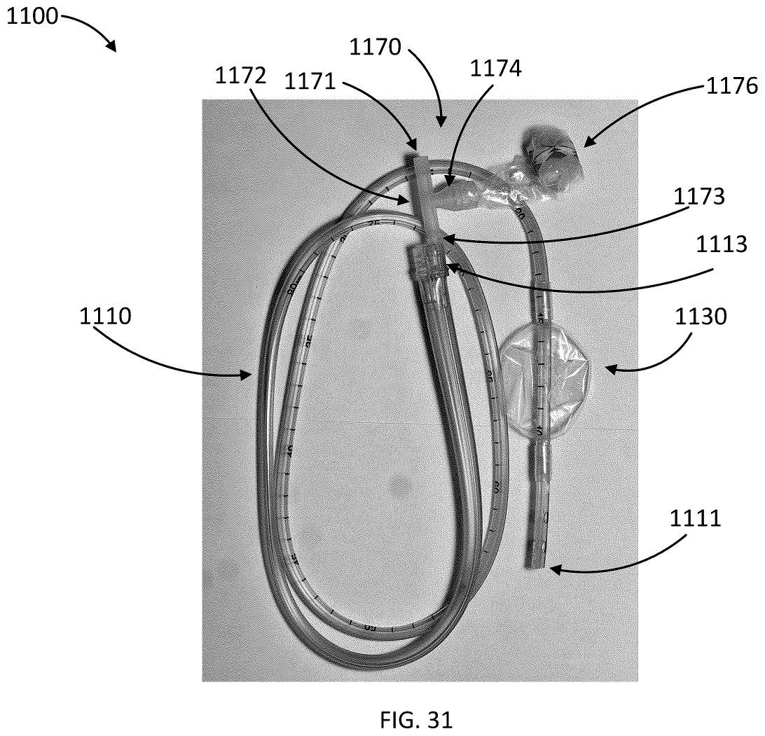

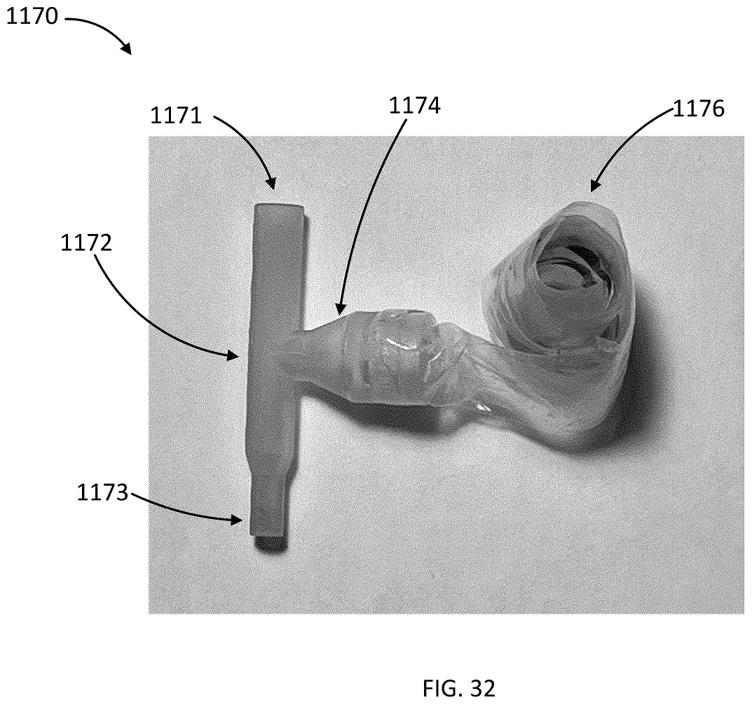

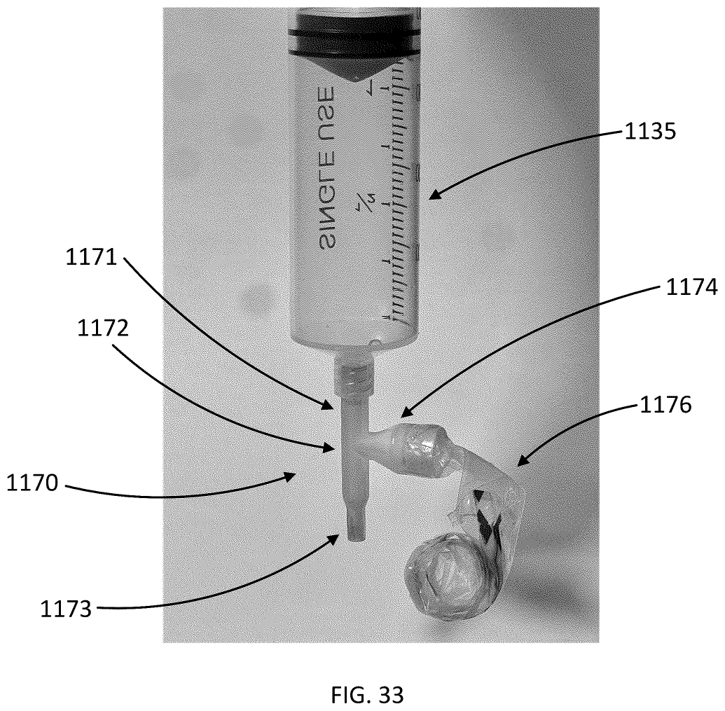

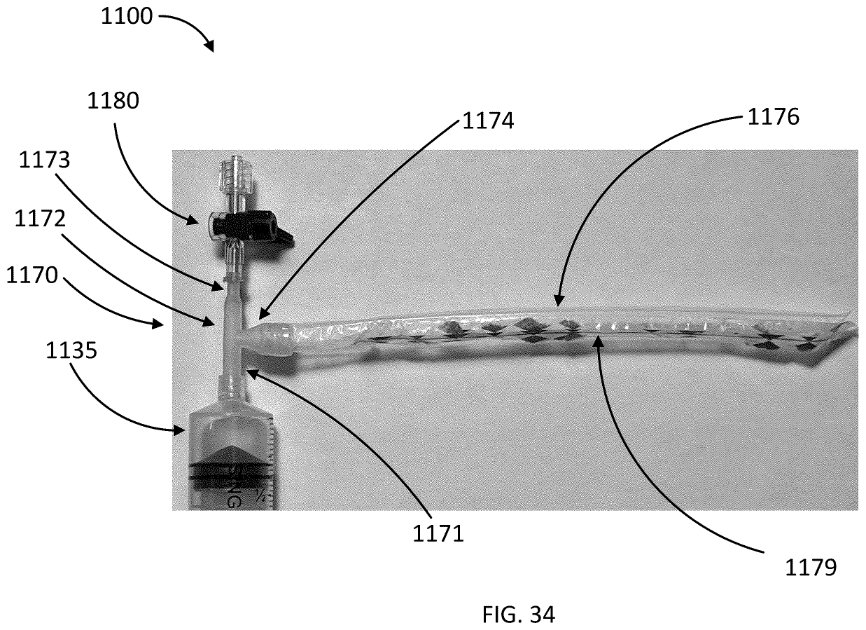

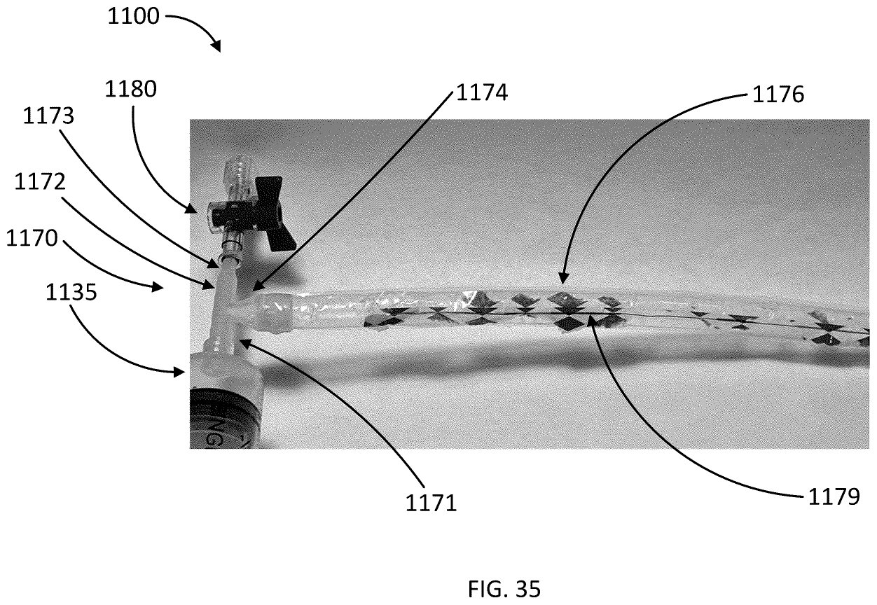

FIGS. 30-35 are schematic illustrations of various portions of a system 1100. Specifically, FIG. 30 is a top view of the system 1100 including a syringe 1135, FIG. 31 is a top view of the system 1100 without the syringe 1135 shown, and FIG. 32 is a top view of a visual indicator 1170 of the system 1100. FIG. 33 is a top view of the syringe 1135 coupled to the visual indicator 1170, with the visual indicator 1170 in an unexpanded configuration. FIGS. 34 and 35 are a top view and a perspective view, respectively, of the visual indicator 1170 coupled to the syringe 1135 and a Luer connector 1180, with the visual indicator 1170 in an expanded configuration.

The system 1100 can be the same or similar in structure and/or function to any of the systems described herein, such as the system 800. For example, the visual indicator 1170 and the syringe 1135 of the system 1100 can be the same or similar in structure and/or function to any of the visual indicators and syringes described herein, such as the visual indicator 870 and the fluid and/or vacuum source 835, respectively.

Furthermore, the system 1100 includes an elongated tube 1110, which can be the same or similar in structure and/or function to any of the elongated tube described herein, such as the elongated tube 810. For example, the elongated tube 1110 includes a first end 1111 (also referred to as a distal end), a second end 1113 (also referred to as a proximal end), and an internal inflatable member 1130. The elongated tube 1110 can define an inflation lumen extending from the second end 1113 to the internal inflatable member 1130 and an additional lumen (e.g., a feeding lumen) extending from the second end 1113 to openings 1114 near the first end 1111.

For example, the visual indicator 1170 includes a housing 1172 and an external inflatable member 1176. The housing 1172 includes an inlet 1171, an outlet 1173, and a port 1174, and defines an interior space. The inlet 1171 can be configured to be coupled to the syringe 1135 via any suitable coupling or fitting, such as a Luer connection. The outlet 1173 can be configured to be coupled to the proximal end 1113 of an elongated tube 1110 such that the interior space is in fluid communication with the inflation lumen of the elongated tube 1110. For example, the outlet 1173 can be disposed within a proximal end of the lumen of the elongated tube 1110.

The inflatable member 1176 (e.g., a balloon) is coupled to the port 1174 and fluidically coupled to the inlet 1171 and the outlet 1173 via the interior space. The inflatable member 1176 can transition between the unexpanded configuration (also referred to as a first, uninflated configuration) and the expanded configuration (also referred to as a second, inflated configuration) in response to an increase in pressure within the interior space caused by the internal inflatable member 1130 being constrained from expanding. FIGS. 34 and 35 show the outlet 1173 coupled to the Luer connector 1180 including a stopcock valve with the valve in the closed configuration to demonstrate the effect of the diversion of fluid delivered from the syringe 1135 into the interior space of the housing 1172 of the visual indicator 1170 into the external inflatable member 1176 to transition the external inflatable member 1176 to an expanded configuration.

Although shown as being coiled in the unexpanded configuration and tubular in the expanded configuration, the inflatable member 1176 can have any suitable shape in each of the unexpanded configuration and the expanded configuration that is associated with easy visual recognition by the user that the inflatable member 1176 has transitioned between the unexpanded and the expanded configuration.

Although not visible in FIGS. 30-35, the visual indicator 1170 can include a valve disposed within the port 1174 and configured to selectively allow fluid to flow between the interior space of the housing 1172 and the inflatable member 1176 (e.g., when a pressure of fluid within the interior space is above a threshold pressure).

The external inflatable member 1176 can include a coil 1179 configured to transition between a coiled configuration associated with the shape of the external inflatable member 1176 in FIGS. 30-33 and an elongated, substantially straight configuration associated with the shape of the external inflatable member 1176 in FIGS. 34 and 35 depending on whether the external inflatable member 1176 is unexpanded or expanded. The coil 1179 can be biased toward the coiled configuration such that, when the external inflatable member 1176 is in the unexpanded configuration, the external inflatable member 1176 forms a coiled shape under the control of the coil 1179 as shown in FIGS. 30-33. The coil 1179 can be disposed along and coupled to the outer surface of the external inflatable member 1176 (e.g., via adhesive).

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. An apparatus, comprising:
an elongated tube having a first end, a second end, and defining a lumen extending from the second end to a location at or near the first end of the elongated tube, the elongated tube defining at least one opening in fluid communication with the lumen such that, when the first end of the elongated tube is disposed in a cavity of a patient and the second end of the elongated tube is disposed outside of the patient, fluid can be delivered from the second end of the elongated tube into the cavity via the lumen and the at least one opening; and
a position indicator coupled to the elongated tube and disposed sufficiently close to the first end such that, when the first end is properly disposed in the cavity of the patient, the position indicator is disposed in the cavity of the patient, the position indicator configured to produce an indication perceivable outside of the patient that the position indicator is disposed in the cavity of the patient, the indication being an indication differentiable from a sound naturally generated by the cavity of the patient.

2. The apparatus of claim 1, wherein the indication is an audible indication.

3. The apparatus of claim 1, wherein the second end of the elongated tube is configured to be coupled to a source of fluid in fluid communication with the position indicator, the indication being a tactile indication experienced via interaction with the source of pressure.

4. The apparatus of claim 2, wherein the position indicator includes a whistle feature, the audible indication being a whistle.

5. The apparatus of claim 1, wherein the position indicator include an electronic sensor configured to sense a characteristic of fluid in the cavity and generate the indication based on a presence of the sensed characteristic.

6. The apparatus of claim 4, further comprising an inflatable member coupled to the elongated tube and having an interior in fluid communication with an interior of the whistle feature, the inflatable member configured to receive fluid from the whistle feature and transition between an unexpanded and an expanded configuration.

7. The apparatus of claim 6, wherein the cavity is the patient's stomach, the inflatable member having a maximum dimension in a plane including the elongated tube in the expanded configuration such that the inflatable member can sufficiently expand in the stomach for the whistle feature to generate the whistle, but such that the inflatable member is restrained from sufficiently expanding for the whistle feature to generate the whistle when the inflatable member is disposed in an esophagus of the patient and in a trachea of the patient.

8. The apparatus of claim 4, wherein the lumen is a feeding lumen, the elongated tube defining a whistle lumen extending from the second end of the elongated tube to the whistle feature and in fluid communication with the whistle feature.

9. The apparatus of claim 8, wherein the elongated tube defines a third lumen extending from the second end to a location at or near the first end of the elongated tube.

10. The apparatus of claim 1, wherein one or more magnetic members are disposed within the elongated tube between the lumen and the first end of the elongated tube, the at least one opening disposed proximally of the one or more magnetic members relative to the first end of the elongated tube.

11. A method, comprising:
producing a fluid flow through a lumen of an elongated tube such that the fluid flow interacts with a position indicator coupled to the elongated tube and disposed within a patient, and
determining whether the position indicator generated an indication that the position indicator is disposed within a target cavity of the patient in response to the fluid flow, the indication being an indication differentiable from a sound naturally generated by the cavity of the patient.

12. The method of claim 11, wherein the determining includes listening for whether an audible indication is emitted from a portion of a body of the patient associated with the target cavity.

13. The method of claim 11, wherein the position indicator includes an inflatable member, the producing including applying pressure to a source of the fluid flow, the source being in fluid communication with the inflatable member via the lumen, and the determining including sensing a resistance of the source of the fluid flow to the applied pressure.

14. The method of claim 11, further comprising, upon determining that the position indicator is disposed in the target cavity, providing liquid fluid through a feeding lumen of the elongated tube to the target cavity.

15. The method of claim 11, further comprising, upon determining that the position indicator is not disposed in the target cavity, manipulating the elongated tube to relocate the position indicator.

16. The method of claim 15, wherein determining that the position indicator is not disposed in the target cavity includes determining that the indication was not produced.

17. A method, comprising:
producing a fluid flow through a lumen of an elongated tube such that the fluid flow interacts with an inflatable member coupled to the elongated tube and disposed within a patient, the inflatable member configured to be inflated via the lumen, and
determining whether the inflatable member is disposed within a non-target cavity or passageway of the patient based on a resistance of the inflatable member to the fluid flow.

18. The method of claim 17, wherein the producing includes applying pressure to a source of the fluid flow, the source being in fluid communication with the inflatable member via the lumen, and the determining including sensing a resistance of the source of the fluid flow to the applied pressure.

19. The method of claim 17, further comprising, upon determining that the inflatable member is not disposed in the non-target cavity or passageway, providing liquid fluid through a feeding lumen of the elongated tube to a target cavity.

20. The method of claim 17, further comprising, upon determining that the inflatable member is disposed in the non-target cavity or passageway, manipulating the elongated tube to relocate the inflatable member.

21.-27. (canceled)
